(19) United States
(12) Patent Application Publication
(10) Pub. No.: US 2020/0297999 A1
(43) Pub. Date: Sep. 24, 2020

(54) SYSTEMS AND METHODS FOR APPLYING ELECTRICAL ENERGY TO TREAT PSORIASIS

(71) Applicant: Thync Global, Inc., Los Gatos, CA (US)

(72) Inventor: Sumon K. PAL, Boston, MA (US)

(21) Appl. No.: 16/842,586

(22) Filed: Apr. 7, 2020

Related U.S. Application Data
(63) Continuation of application No. 15/983,885, filed on May 18, 2018.
(60) Provisional application No. 62/509,603, filed on May 22, 2017.
(60) Provisional application No. 62/522,054, filed on Jun. 19, 2017.
(60) Provisional application No. 62/522,629, filed on Jun. 20, 2017.
(60) Provisional application No. 62/598,462, filed on Dec. 13, 2017.

(51) Int. Cl. A61N 1/32; A61N 1/04; A61N 1/36

(52) U.S. Cl. CPC A61N 1/328; A61N 1/0476; A61N 1/0408; A61N 1/048; A61N 1/36034; A61N 1/0492; A61N 1/0456

(57) ABSTRACT

Methods and apparatuses for treating a medical disorder by the application of non-invasive electrical stimulation. The applied electrical energy may cause autonomic nervous system (ANS) neuromodulation. In general, described herein are methods for electrical energy to a subject, and particularly to the subject's neck with an electrical waveform adapted to improve the medical disorder. Specifically, described herein are methods and apparatuses for treating a patient having psoriasis by non-invasively applying electrical energy.

### CROSS REFERENCE TO RELATED APPLICATIONS

This patent application is a continuation of U.S. patent application Ser. No. 15/983,885, filed on May 18, 2018 (titled “SYSTEMS AND METHODS FOR APPLYING ELECTRICAL ENERGY TO TREAT PSORIASIS”); which claims priority to U.S. Provisional Patent Application No. 62/509,603, filed May 22, 2017 (titled “SYSTEMS AND METHODS FOR TRANSDERMAL ELECTRICAL STIMULATION TO TREAT PSORIASIS”); U.S. Provisional Patent Application No. 62/522,054, filed Jun. 19, 2017 (titled “SYSTEMS AND METHODS FOR TRANSDERMAL ELECTRICAL STIMULATION TO TREAT PSORIASIS”); U.S. Provisional Patent Application No. 62/522,629, filed Jun. 20, 2017, titled “SYSTEMS AND METHODS FOR TRANSDERMAL ELECTRICAL STIMULATION TO TREAT MEDICAL DISORDERS”); and U.S. Provisional Patent Application No. 62/598,462, filed Dec. 13, 2017, titled “SYSTEMS AND METHODS FOR TRANSDERMAL ELECTRICAL STIMULATION TO TREAT MEDICAL DISORDERS,” each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

This invention relates generally to methods and apparatuses for noninvasive neuromodulation to treat a disorder such as a skin disorder, including inflammatory skin disorders (e.g., inflammatory skin disorder), and more specifically to apparatuses and methods for non-invasive electrical stimulation adapted to treat medical disorders such as psoriasis.

The present invention relates to methods and systems for non-invasively applying electrical energy to treat a skin disorder such as, but not limited to psoriasis. In some variations these methods and apparatuses may be configured to prevent the disorder (such as psoriasis). For example, described herein are methods for treating psoriasis or its associated symptoms using non-invasive electrical energy applicator systems worn on the body. In particular described herein are wearable, non-invasive, electrical energy applicator (e.g., neurostimulator) apparatuses, configured to be applied to the user (e.g., the user's neck and/or head and/or neck/upper back) to treat psoriasis.

### BACKGROUND

Many medical disorders would benefit from non-invasive and non-pharmacological treatments. In particular, skin disorders, including inflammatory skin disorders such as psoriasis. Psoriasis is a common, chronic recurring condition characterized by the eruption of reddish, silvery-scaled maculopapules, which occur predominantly on the elbows, knees, scalp, and trunk. Skin rapidly grows and accumulates at psoriatic plaques, i.e., red scaly patches.

The etiology of psoriasis is not fully understood, but it appears that stress is considered to play an important role in the onset and exacerbation of psoriasis. Normal physiologic response to stress involves activation of the hypothalamus-pituitary-adrenal (HPA) axis and sympathetic adrenomedullary (SAM) axis, both of which interact with immune functions. Generally, in normal individuals, stress elevates stress hormones (i.e., increases cortisol). However, according to available studies, exposure to stress in psoriatic patients has been associated with diminished HPA responses and upregulated SAM responses.

Psoriasis is difficult to treat. Currently available treatments for psoriasis are of limited effectiveness in many patients and, generally, can be used only for a limited duration. For example, topical treatments can often irritate normal skin, cannot be used for long periods, and may cause an aggressive recurrence of the condition when the treatment stops. Phototherapy can improve psoriasis in some, but not all, patients. Photochemotherapy, i.e., the combined therapy of psoralen and ultraviolet A phototherapy (PUVA), has also been used to treat psoriasis. However, PUVA is associated with nausea, headache, fatigue, burning, itching. Long-term PUVA treatment is associated with squamous cell carcinoma. Psoriasis can also be treated by systemic treatment, e.g., by injection or oral administration of medications, such as methotrexate, cyclosporine and retinoids. However, these medications are known to have toxic side effects, thus cannot be used too frequently. Patients undergoing systemic treatment are required to have regular blood and liver function tests, and pregnancy must be avoided for the majority of these treatments. Most people experience a recurrence of psoriasis after systemic treatment is discontinued. Biologics, such as AMEVIVE, ENBREL, HUMIRA, and REMICADE AND RAPTIVA, are relatively new therapies that focus on specific aspects of the immune function leading to psoriasis. However, the long-term impact of the biologics on immune function is unknown and they are very expensive and only suitable for very few patients with severe psoriasis.

Non-invasive neuromodulation, typically by the application of transdermal electrical stimulation (TES), e.g., applied through scalp electrodes, has been used to affect brain function in humans. TES has been used therapeutically in various clinical applications, including treatment of pain, depression, epilepsy, and tinnitus. Despite the research to date on TES neuro stimulation, the Applicants are not aware of any methods or apparatuses applying non-invasive electrical energy (e.g., neuromodulatoin) to treat a skin disorder such as psoriasis.

Thus, in general, it would be advantageous to provide apparatuses and methods for non-invasively applying electrical energy for treatment of a medical disorder such as psoriasis. The methods and apparatuses described herein may address these needs.

### SUMMARY OF THE DISCLOSURE

The present invention relates to methods and apparatuses for treating disorders, including (but not limited to) psoriasis. In general, these methods may include non-invasively applying electrical energy (e.g., from a wearable electrical energy applicator, e.g., neurostimulator and/or neuromodulation applicator) to the subject, and applying appropriate non-invasive electrical stimulation for a treatment period of longer than 1 minute (e.g., longer than 5 minutes, longer than 10 minutes, longer than 15 minutes, between 5 minutes and 2 hours, between 5 minutes and 1 hour, etc.) once daily, or more than once daily (e.g., 2× daily, 3× daily, 4× daily, 5× daily, etc.) or every other day, every third day, etc.

Although the disorders described herein are typically inflammatory medical disorders, other inflammatory and/or other skin disorders may be treated using any of the apparatuses and methods described herein. For example, other inflammatory (and/or autoimmune) disorders that may be treated include: rheumatoid arthritis, inflammatory bowel disease, multiple sclerosis, Sjogren's syndrome, Graves' or Hashimoto's thyroiditis, asthma and/or lupus. Other skin-specific disorders that may be treated include, but are not limited to: Pruritus (Itch), Hyper-hidrosis (excessive sweating), facial erythema (facial flush), atopic dermatitis, eczema, prurigo nodularis, lichen planus, chronic urticarial, alopecia areata, rosacea and/or vitiligo. Other medical disorders may include migraines. Although the examples described herein focus primarily on psoriasis, the methods and apparatuses described herein may be used to treat any of the disorders discussed above.

Without being bound by any particular theory of operation, the methods and apparatuses described herein may be referred to as non-invasive autonomic nervous system (ANS) neuromodulation apparatuses and/or methods, or simply neuromodulation apparatuses and methods. The non-invasive electrical energy applied herein may target peripheral nerves and utilize these pathways to influence brain function; by delivering pulsed electrical currents to specific nerve pathways, biochemical and biometric data has shown a significant suppression of basal sympathetic tone and lower stress. Surprisingly this method has also resulted in a reduction in the severity (e.g., reduction in plaque/maculopapules number and/or size) of psoriasis maculopapules/plaques. As stated above, psoriasis patients are believed to have an upregulated sympathetic response which is directly correlated to the severity of their condition. Without being bound by a particular theory, the methods described herein for the application of electrical energy (e.g., non-invasive ANS neuromodulation) may lower sympathetic tone in individuals with psoriasis thereby improving their condition. The lack of side effects using the application of non-invasive electrical stimulation (e.g., ANS neuromodulation) described herein makes it particularly advantageous as compared to current methods of treatment of psoriasis. Although preliminary evidence suggests that the effective electrical stimulation causes neuromodulation, and in particular, causes ANS neuromodulation, it is possible that the electrical stimulation is acting in part or entirely via a different biological mechanism. Regardless of the underlying mechanism of action, the methods and apparatuses below are effective (using the parameters described herein) for treating inflammatory skin disorders, including psoriasis. Any of the electrical energy applying apparatuses described herein may be referred to as neuromodulation apparatuses and/or as ANS neuromodulation apparatuses.

As used herein, the term “noninvasive” or “noninvasively” may refer to externally applied (e.g., via skin or mucus-membrane contact) without cutting the body, e.g., skin. Although the electrical energy applied by the methods and apparatuses described herein may be applied noninvasively, the energy may penetrate into the tissue; the term “noninvasive electrical energy” or “noninvasive neuromodulation” may refer to the point of application of the electrical energy (e.g., on the skin) and not the point of effect of the electrical energy.

A non-invasive electrical energy applicator may be applied by the patient herself, and in some variations the patient may manually adjust one or more of the electrical waveform parameters to enhance comfort. The attachment sites for the electrodes may include at least one location on the neck and may also include a second location on the subject's head or neck (e.g., back of the neck). Alternatively two electrode locations may be on the neck/upper back; one electrode location may be on the subject's neck (over the C1-C7 region) and a second electrode location may be below the neck (upper back, e.g., over the C4-T2 region); or two electrodes may be on the subject's skin below the neck (e.g., within the C5-T2 region, etc.).

For example, a method of non-invasively treating psoriasis may include attaching a first electrode to a subject's neck at a first location and a second electrode to the subject's head or neck at a second location, wherein the first and the second electrode are coupled to a non-invasive electrical energy (e.g., neuromodulation and/or ANS neuromodulation) applicator worn by the subject. Once applied, the non-invasive electrical energy applicator may be used to apply an electrical energy (e.g., electrical stimulation, neurostimulation, neuromodulation) between the first and second electrodes for a stimulation duration. The applied electrical stimulation may be an ‘ensemble waveform’ as described herein and described in U.S. application Ser. No. 14/715,476, filed May 18, 2015 (now Publication No. US-2015-0328461), previously incorporated by reference in its entirety. For example, the electrical stimulation may have a peak amplitude of greater than 3 mA, a frequency of greater than 250 Hz, and a duty cycle of greater than 10%. The application of the electrical stimulation may be continued for a stimulation duration of at least one minute. For example, the stimulation duration (the time during which the non-invasive neuromodulation waveform is being applied by the applicator) may be between 1 minute and 120 minutes, between 1 minute and 90 minutes, between 1 minute and 60 minutes, etc., or may be between any lower value (where the lower value may be 0.5, 1, 2, 3, 4, 5, 6, 7, 8, 9, 10, 11, 12, 13, 14, 15, 20, 25, 30, 35, 40, 45, 50, 55, 60, 75, 90, 105, 120, etc. minutes) and an upper value (where the upper value may be 2, 3, 4, 5, 6, 7, 8, 9, 10, 11, 12, 13, 14, 15, 20, 25, 30, 35, 40, 45, 50, 55, 60, 75, 90, 105, 120, 150, etc. minutes), and the lower value is always lower than the upper value.

The wearable non-invasive electrical energy applicator may be attached by any appropriate method, including adhesively attaching, attaching using a strap, attaching via a garment such as a hat, band, etc., attaching via a bandage or wrap, or the like. As mentioned, the first electrode may be attached to the subject's neck. The first electrode may be on or attached directly to the body of the wearable non-invasive electrical energy applicator. The second electrode may also be attached to the subject's head or neck; for example, the second electrode may be attached to the subject's neck above the subject's vertebra prominens.

Any of these methods may allow the patient's physician (who may also be referred to as the user) to select a set of parameters for the electrical stimulation to be applied. Any individual or combination of parameters may be modulated/set by the user, and this modulation may be performed before and/or during the application of the stimulation. For example, a user (e.g., physician) may modify one or more parameters such as: stimulation duration, frequency, peak amplitude, duty cycle, capacitive discharge on or off, and DC offset. The adjustment may be made within a fixed/predetermined range of values providing for different doses (e.g., for frequency, the user may adjust the frequency between a minimum value, such as 250 Hz, and a maximum value, such as 40 kHz, or any sub-range therebetween). The non-invasive neuromodulation applicator may be worn (and energy applied) while the subject is awake and/or while the subject sleeps. The subject may also be referred to as a patient, and may be any human or non-human (including non-human primates).

Examples of non-invasive neuromodulation ensemble waveforms that may be appropriate for treating psoriasis are described in greater detail below. In general, these non-invasive neuromodulation ensemble waveforms may be monophasic or biphasic (or both during different periods); in particular non-invasive neuromodulation ensemble waveforms may include biphasic electrical stimulation. This biphasic electrical stimulation may be asymmetric with respect to positive and negative going phases. Psoriasis-treating non-invasive electrical waveforms may also have a duty cycle (e.g., time on relative to time off) of between 10% and 90%, e.g., a duty cycle of between 30% and 60%. The peak amplitude of the applied current may also be controlled. In general, the peak amplitude may be greater than 3 mA (greater than 4 mA, greater than 5 mA, greater than 6 mA, greater than 7 mA, greater than 8 mA, etc. or between about 3 mA and about 30 mA, between 3 mA and 20 mA, between 5 mA and 30 mA, between 5 mA and 20 mA, etc.).

As mentioned above, any of the electrical energy parameters (e.g., peak current amplitude, frequency, DC offset, percent duty cycle, capacitive discharge, etc.) may be changed during the ensemble waveform, so that sub-periods of different parameters may be consecutively applied. The frequency may be between 250 Hz and 50 kHz (e.g., a minimum of: 250, 300, 350, 400, 450, 500, 550, 600, 650, 700, 750, 800, 850, 900, 950, 1000, 1500, 2000, 3000, 4000, 5000, etc. Hz and a maximum of 350, 400, 450, 500, 550, 600, 650, 700, 750, 800, 850, 900, 950, 1000, 1500, 2000, 3000, 4000, 5000, 6000, 7000, 8000, 9000, 10000, 12000, 15000, 20000, 25000, 30000, 35000, 40000, 50000 Hz, where the minimum is always less than the maximum).

As mentioned, any appropriate electrical energy (e.g., “stimulation” or “neuromodulation”) duration may be used. For example, the step of continuing application of the electrical stimulation for a stimulation duration may include continuing for a stimulation duration of at least five minutes.

Any of the non-invasive neuromodulation ensemble waveforms described herein may be modulated by amplitude modulation, using an appropriate AM carrier frequency. For example, applying the non-invasive neuromodulation waveform(s) may comprise applying electrical stimulation having amplitude modulation, and the amplitude modulation may generally have a frequency of less than 250 Hz (e.g., between 0.01 Hz and 250 Hz, 1 Hz and 250 Hz, 5 Hz and 200 Hz, 10 Hz and 200 Hz, etc.).

In some variations, applying the non-invasive neuromodulation psoriasis-treating ensemble waveform may include applying electrical stimulation having a burst mode. A bursting mode may include periods where the non-invasively applied neuromodulation stimulation is quiescent (“off”). Note that although the majority of the examples described herein include the use of ensemble waveforms in which one or more (though often just one) stimulation parameter changes during different, predefined component waveforms that are sequentially applied as the ensemble waveform, in some variations only a single component waveform is applied. Similarly, a component waveform may vary continuously or discretely (by steps) for one or more component waveforms.

For example, described herein are methods of non-invasively treating psoriasis that may include: placing a first electrode and second electrode of a wearable non-invasive neuromodulation applicator on a subject's body; activating the wearable non-invasive neuromodulation applicator to deliver a biphasic electrical stimulation between the first and second electrodes having a duty cycle of greater than 10 percent, a frequency of 250 Hz or greater, and an intensity of 3 mA or greater, wherein the biphasic electrical stimulation is asymmetric with respect to positive and negative going phases; and reducing repeating the placing and activating steps to reduce psoriasis.

Any of the methods of non-invasively applying electrical energy for treating psoriasis described herein may be used in conjunction with, and may surprisingly enhance, pharmaceutical treatments of psoriasis. In particular, when a subject is co-treated with both a pharmaceutical treatment (e.g., a biologic such as AMEVIVE, ENBREL, HUMIRA, AND REMICADE and RAPTIVA), the effect of the biological may be accelerated. In addition, lower doses may be effectively used.

In some variations, the methods described herein may be configured to apply a dose of electrical energy that is predetermined and/or optimized for treating psoriasis; the patient may be prevented from adjusting the dosage.

In any of these methods, the first step may be identifying a subject suffering from psoriasis. Psoriasis may be diagnosed by any method known in the art, including by identifying maculopapules/plaques on the patient's skin. The therapy may be provided at regular (e.g., daily, multiple times daily, every other day) until an appropriate response is seen, including a reduction in maculopapule/plaque size and/or frequency (e.g., a 5% or greater reduction, a 10% or greater reduction, a 15% or greater reduction, a 20% or greater reduction, a 25% or greater reduction, a 30% or great reduction, a 40% or greater reduction, a 50% or greater reduction, a 60% or greater reduction, a 70% or greater reduction, an 80% or greater reduction, a 90% or greater reduction, a 95% or greater reduction, etc.).

For example, a method of non-invasively applying electrical energy to treat psoriasis may include: placing a first electrode of a wearable no electrical energy (e.g., neuromodulation or in some variations ANS neuromodulation) applicator on the subject's skin on the subject's temple region and a second electrode on a back of the subject's neck above a vertebra prominens; treating psoriasis by activating the wearable electrical energy applicator to deliver a biphasic electrical stimulation having a duty cycle of greater than 10 percent, a frequency of 250 Hz or greater, and an intensity of 3 mA or greater, wherein the biphasic electrical stimulation is asymmetric with respect to positive and negative going phases; and treating psoriasis by applying the biphasic electrical stimulation between the first and the second electrodes for 10 seconds or longer.

A method of treating psoriasis in a subject in need thereof may include: placing a first electrode of a wearable electrical energy applicator on the skin of a subject having psoriasis at the back of the subject's neck (e.g., on a back of the subject's neck above a vertebra prominens) and the placing the second electrode on the subject's neck or head; activating the wearable electrical energy applicator to deliver a biphasic electrical stimulation having a duty cycle of greater than 10 percent, a frequency of 250 Hz or greater, and an intensity of 3 mA or greater, wherein the biphasic electrical stimulation is asymmetric with respect to positive and negative going phases; and treating the subject's psoriasis by applying the biphasic electrical stimulation between the first and second electrodes for 5 minutes or longer.

Any of the method components described above may be incorporated into any of these exemplary methods as well. For example, attaching the electrical energy applicator and/or electrodes may refer to adhesively attaching, mechanically attaching or the like. In general, the electrical energy applicator may be applied directly to the body (e.g., coupling the body to the skin or clothing of the patient directly) or indirectly, e.g., attaching to the body only by coupling with another member (e.g., electrode) that is already attached or attachable to the body. The attachment location may be independent of the location of one or more maculopapules and/or plaques on the subject's skin.

In any of the methods described herein, the user may be allowed and/or required to select the waveform ensemble from a list of possible waveform ensembles, which may be labeled to indicate name, content, efficacy, and/or the like. Alternatively or additionally, the user may be prevented from selecting or altering the waveform(s). In some variations, the subject may be permitted or allowed (e.g., using a wearable electronic and/or handheld electronic apparatus) to modify or adjust the intensity of the electrical stimulation to be applied.

The electrodes and electrical energy applicator may be worn while the subject sleeps, or prior to sleeping.

Any of the methods described herein may be automatically or semi-automatically controlled, and may include processing of feedback from any of the sensors to regulate the application of electrical energy, including modifying one or more electrical waveform parameter based on the sensed values.

In any of these variations, the apparatus may be specifically adapted for comfort, convenience or utility when used with a subject's suffering from psoriasis. For example, in apparatuses in which there is a visible psoriatic plaque.

Although the stimulation parameters may be adjusted or modified by a user, e.g., a prescribing physician or health care provider, the subject (patient) wearing the apparatus may not adjust the stimulation parameters, but may control or adjust the time of non-invasively applied electrical energy, such as the time of day and/or the intensity of the stimulation, stopping/restarting the stimulation, etc. Any of these method may include modifying, by a party that is not the subject (e.g., the user), a stimulation parameter of the wearable electrical energy application device (e.g., neuromodulator), wherein the stimulation parameter includes one or more of: stimulation duration, frequency, peak amplitude, duty cycle, capacitive discharge, DC offset, etc. For example, the user (patient's physician) may adjust the dose prescribed and available for delivery to the patient, which may be controlled by the electrical energy application apparatus.

Any of these methods may also include automatically stopping, starting or modulating the wearable neuromodulation applicator per a physician-provided prescription. For example, in some variations, the subject (patient) may start/stop or adjust the intensity (e.g., amplitude) of a preset electrical energy waveform within a pre-defined range.

In operation, the wearable electrical energy applicator may automatically or manually triggered to deliver the biphasic electrical stimulation. The apparatus may also be configured to transmit a notification (directly or via a user computing device) that reminds the subject to wear the electrical energy applicator, for example, transmitting a notification that reminds the subject to wear the electrical energy applicator based on input from a location sensor in the non-invasive electrical energy applicator or wirelessly connected to the electrical energy applicator.

The methods described herein may also include providing a metric to the subject showing compliance with the treatment protocol (e.g., regular use for the prescribed time). The methods may include a metric showing improvement based on user-reported and/or quantified (e.g., plaque/maculopapule count and/or size) metrics.

In addition, any of the methods described herein may also include concurrently delivering a calming sensory stimulus when activating the wearable non-invasive neuromodulation applicator, such as concurrently delivering a calming sensory stimulus when activating the wearable non-invasive neuromodulation applicator, wherein the calming sensory stimulus is one or more of auditory stimulus, olfactory stimulus, thermal stimulus, and mechanical stimulus.

Also described herein are wearable electrical energy (e.g., neuromodulation) applicators for treating psoriasis. These apparatuses may be configured to perform any of the methods described herein. In general, these apparatuses may include: a body; a first electrode; a second electrode (the apparatuses may be part of a separate but attachable, e.g., disposable, electrode assembly that couples to the body); and an electrical energy control (e.g., neuromodulation) module at least partially within the body. The electrical energy control module may include a processor, a timer and a waveform generator, and the electrical energy control module may be adapted to deliver an electrical (e.g., biphasic, asymmetric) stimulation signal for a stimulation duration (e.g., 10 seconds or longer) between the first and second electrodes. The electrical stimulation which may be a neuromodulation ensemble waveform, may have a duty cycle of greater than 10 percent, a frequency of 250 Hz or greater, and an intensity of 3 mA or greater, wherein the biphasic electrical stimulation is asymmetric with respect to positive and negative going phases. The wearable neuromodulation applicator may generally be lightweight (e.g., may weigh less than 50 grams, etc.). Any of the electrical energy applicators described herein may be non-invasive neuromodulation applicators, and may include at least one sensor coupled to the body for monitoring the subject (e.g., the subject's sympathetic and/or parasympathetic tone or state).

Any of these apparatuses may include a psoriasis medicament on the treatment pad for jointly treating with a psoriasis medicine.

Any appropriate non-invasive neuromodulation waveform(s) may be used, particularly those that enhance a relative reduction in sympathetic tone, compared to parasympathetic tone. For example, the duty cycle may be between 10% and 90%. The electrical stimulation may have a frequency greater than 250 Hz, 500 Hz, 750 Hz, 5 kHz, etc. For example, the frequency may be between 250 Hz to 50 kHz. The electrical stimulation may comprise amplitude modulation, as discussed above, having a frequency of less than 250 Hz. The non-invasive neuromodulation electrical stimulation may include a burst mode, such as a burst mode having a frequency of bursting that is less than 250 Hz.

The non-invasive neuromodulation waveform(s) may be pre-programmed. The apparatus may include at least one sensor that measures the subject's autonomic function, wherein the measurement of autonomic function may measure one or more of: galvanic skin resistance, heart rate, heart rate variability, or breathing rate. The feedback from the at least one sensor may be used to adjust the stimulation parameters. Ideally, the treatment may be performed to induce a sustained (e.g., greater than 5 minutes, greater than 10 minutes, greater than 15 minutes, greater than 20 minutes, greater than 25 minutes, greater than 30 minute, etc.) upregulated sympathetic response. Based on the sensor detection, the apparatus may increase any of the one or more stimulation parameters, such as: the current, the frequency, the duration, etc., until the subject is experiencing a robust suppression of basal sympathetic tone, and therefore a reduction in stress.

Any of these devices may include a visual indicator (e.g., light, screen, etc., including LED(s), displays, etc.) that is configured to be turned down or turned off when the wearable electrical energy (e.g., neuromodulation) system is activated.

Examples of the methods described herein include methods of treating immune disorders, including (but not limited to) psoriasis by non-invasively applying electrical energy (e.g., in some variations, applying non-invasive ANS neuromodulation). For example, a method of treating an immune disorder such as psoriasis in a subject suffering from the immune disorder by non-invasively applying electrical energy (e.g., neuromodulation) includes: non-invasively applying electrical energy (e.g., neuromodulation) to the subject to reduce one or more of the size and number of psoriasis plaques, wherein the applied electrical energy has a peak amplitude of greater than 3 mA, a frequency of greater than 250 Hz, and a duty cycle of greater than 15%.

A method of treating psoriasis in a subject suffering from psoriasis by non-invasively applying electrical energy may include: non-invasively applying electrical energy to the subject to reduce one or more of the size and number of psoriasis plaques, wherein the electrical energy is applied for a session of at least 5 minutes per day, for at least 8 treatment sessions. The sessions may be performed on sequential days (e.g., every day for 8 days or more) or alternating days (e.g., every other day for 16 days or more; every third day for 24 days or more; every fourth day for 32 days or more, every fifth day for 40 days or more, every sixth day for 48 days or more, every seventh day for 56 days or more, etc.). In some variations, the sessions may be applied on alternating weeks (e.g., one week of 4-7 daily sessions on/one week off, etc.). More than one session may be applied per day. For example, two sessions of 5 minute each may be applied per day, etc. The sessions may have a duration of between 5-90 minutes (e.g., 10 minutes, 12 minutes, 15 minutes, 20 minutes, etc.).

For example, a method of treating psoriasis in a subject suffering from psoriasis by applying electrical energy may include: non-invasively applying electrical energy to the subject to reduce one or more of the size and number of psoriasis plaques, wherein the electrical energy is applied for at least 10 minutes per day, each of 5 or more days a week for at least two weeks (e.g., at least three weeks, at least four weeks, at least five weeks, at least six weeks, at least seven weeks, at least eight weeks, etc.)

For example, a method of treating psoriasis in a subject suffering from psoriasis by applying electrical energy may include: attaching at least one of a pair of electrodes to a region along a midline of a back of the subject's neck; applying electrical energy between the pair of electrodes to reduce one more ore of the size and number of psoriasis plaques.

A method of non-invasively treating an inflammatory and/or a skin disorder may generally include: non-invasively applying electrical energy between a pair of electrodes, wherein at least one electrode of the pair of electrodes is attached to the subject's neck; wherein the applied electrical energy has a peak amplitude of greater than 3 mA, a frequency of greater than 250 Hz, and a duty cycle of greater than 15%; and continuing the application of the electrical energy to induce a decrease in sympathetic tone and thereby reduce the symptoms of the inflammatory and/or skin disorder. The inflammatory and/or skin disorder may be psoriasis; alternatively, the inflammatory and/or skin disorder may be one of: rheumatoid arthritis, inflammatory bowel disease, multiple sclerosis, Sjogren's syndrome, Graves' or Hashimoto's thyroiditis, asthma, lupus, psoriasis, Pruritus (Itch), Hyper-hidrosis (excessive sweating), facial erythema (facial flush), atopic dermatitis, eczema, prurigo nodularis, lichen planus, chronic urticarial, alopecia areata, rosacea, vitiligo and migraines.

In any of the methods described herein, applying may comprise applying electrical energy between a first electrode and a second electrode attached to either or both of the subject's head and neck, wherein the first electrode is attached at a first location and a second electrode is attached at a second location, further wherein the first and the second electrode are coupled to an electrical energy (e.g., neuromodulation) applicator worn by the subject.

In any of the methods described herein, applying may comprise applying the electrical energy to a back of the subject's neck.

In any of the methods described herein, electrical energy may be applied 5 or more days a week at least once per day for at least two weeks. For example, electrical energy (e.g., neuromodulation or “ANS neuromodulation”) may be applied at least once per day for at least 10 minutes each day for at least two weeks (e.g., at least 3 weeks, at least 4 weeks, at least 5 weeks, at least 6 weeks, at least 7 weeks, at least 8 weeks, etc.). Electrical energy may be applied at least once per day for at least 15 minutes each day for at least eight weeks.

In any of the methods described herein, the applied electrical energy may have a peak amplitude of greater than 3 mA, a frequency of greater than 1 kHz, and a duty cycle of 20% or more.

When applying electrical energy to treat psoriasis, applying n electrical energy may further comprise applying the electrical energy to a patient being treated with a drug for psoriasis.

In any of the methods described herein, the method may include determining one or more of the subject's sympathetic tone during the application of electrical energy and adjusting the electrical stimulation (electrical energy) based on the sympathetic tone.

In any of these methods, applying the electrical energy may comprise applying the electrical energy from one or more electrodes attached above the subject's vertebra prominens.

The electrical energy may be a biphasic electrical stimulation, e.g., a biphasic electrical stimulation that is asymmetric with respect to positive and negative going phases.

In any of these methods, applying may comprise non-invasively applying the electrical energy having a duty cycle of between 20% and 90%. For example, applying may comprise applying the electrical energy having a duty cycle of between 20% and 60%. Applying may comprise applying the electrical energy having a peak amplitude of 5 mA or greater. Applying may comprise applying the electrical energy having amplitude modulation. Applying may comprises applying the electrical energy having amplitude modulation, and further wherein the amplitude modulation has a frequency of less than 250 Hz.

Also described herein are wearable non-invasive neuromodulation apparatus configured to treat an immune disorder, including psoriasis, by the non-invasive delivery of electrical energy. In general, these apparatuses (which may be systems and/or devices) may include a non-invasive neuromodulation applicator that is wearable and a set of software or firmware instructions that are executed by a wireless communications device (e.g., smartphone, tablet, etc.) that control dosing by the device. For example, described herein are apparatuses comprising: a first electrode and a second electrode; a controller configured to apply a non-invasive neuromodulation waveform between the first and second electrodes, wherein the non-invasive neuromodulation waveform has a peak amplitude of greater than 3 mA, a frequency of greater than 250 Hz, and a duty cycle of greater than 15%; and a computer readable medium having a set of computer-readable instructions recorded thereon, the computer-readable instructions, when executed by a processor, cause the processor to: apply a dosing regimen from the controller wherein the dosing regimen spans multiple days (e.g. the dosing regimen may be, for example, applying the non-invasive neuromodulation for at least 10 minutes per day, each of 5 or more days a week for at least two weeks).

The first and second electrodes may include gel pad (or electrode assemblies) that connect, via an electrical connector, to the controller. The first and second electrode are adapted to be worn along the midline of a back of a neck. For example, the first and second electrode may be spaced apart from each other on a substrate so that they are between 0.2 and 2.5 inches apart (on center).

Any of these apparatuses may be configured to be worn on the neck. For example, the apparatus may include a neckband configured to be worn around a subject's neck, wherein the neckband comprises an electrode alignment guide region (e.g., on a portion of the neckband configured to be worn on the back of the neck) that is adapted/configured to couple to the first and second electrodes. This may include one or more connectors (snaps, etc.) to which the electrode assembly including the first and second electrodes (e.g., gel pad) can electrically couple. The neck band may also include a dock configured to couple to the controller (e.g., a housing enclosing the controller) that makes electrical connection to the controller and an electrical line (e.g., electrical trace, wire, etc.) within or on the neck band. This electrical line also connects to the electrodes through the electrode alignment guide. The dock may be on a front portion of the neckband.

The dosing regimen may be configured to non-invasively apply electrical energy (e.g., neuromodulation) at least once per day for at least 10 minutes each day for at least two weeks (e.g., at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 14 weeks, at least 16 weeks, at least 18 weeks, etc.). For example, the dosing regimen may be configured to non-invasively apply neuromodulation at least once per day for at least 15 minutes each day for at least three weeks (e.g., at least 4 weeks, at least 5 weeks, at least 6 weeks, at least 7 weeks, at least 8 weeks, at least 9 weeks, at least 10 weeks, at least 11 weeks, at least 12 weeks, at least 14 weeks, at least 16 weeks, at least 18 weeks, etc.). The computer-readable instructions may be configured to display a user interface that allows the user to start and/or stop the dose being delivered. The computer-readable instructions may also cause the processor to track the operation of the apparatus, including the delivery of the dose(s).

In any of these apparatuses, the controller may be enclosed in a housing. The housing may also include two (or more) electrical connectors configured to electrically connect to the first and second electrodes, respectively. The housing may include a button or other control for turning the device on/off. In variations in which the neckband is used, the housing may be configured to mate with the dock on the neckband. If a neckband is not included, the housing may be configured to attach to the electrode assembly (gel pad) including the first and second electrodes.

The housing and enclosed electronics (e.g., controller, battery, indicator/LEDs, wireless communication circuitry, etc.) may be relatively small and lightweight. For example, the housing and enclosed components may weigh less than 50 g.

In any of these apparatuses, the controller may be configured to non-invasively apply electrical energy (e.g., neuromodulation, ANS neuromodulation, etc.) having a peak amplitude of greater than 3 mA, a frequency of greater than 1 kHz, and a duty cycle of 20% or more.

In any of these apparatuses, the controller may be further configured to prevent the device from delivering electrical energy at 15% duty cycle or less (e.g., non-therapeutic electrical energy). Alternatively or additionally the controller may be configured to prevent the device from delivering a charge per phase below a predetermined threshold.

The computer readable medium may be configured to operate on a smartphone.

Any of these apparatuses may include (e.g., as a part of or in communication with the controller), a wireless communication circuit configured to wirelessly communicate between the controller and the processor executing the computer-readable instructions. The wireless communication circuit may be configured to operate in Bluetooth, Wi-Fi, etc.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawing.

FIG. 1 schematically illustrates a base waveform which may be repeated and modified according to waveform parameters to form component waveforms which may be combined to form ensemble waveforms, as described herein.

FIGS. 2A-2F show electrode positions for one configuration (“Configuration 3”) on a model user head that may be used with the methods and apparatuses of treating psoriasis, as described herein.

FIG. 3A illustrates one example of a neurostimulator that may be configured for use with (and may deliver) the ensemble waveforms described herein.

FIGS. 3B-3G illustrate another example of a neurostimulator as described herein.

FIGS. 3H-3K illustrate a first example of one variation of an electrode assembly.

FIG. 3L illustrates the application of an electrode assembly that may be worn on the subject's head and neck to treat psoriasis.

FIG. 3M illustrates the neurostimulator device worn on the subject's head and neck.

FIG. 4A shows an example of an adhesive electrode pad configured to be worn over the cervical and thoracic region (on the patients neck) having a pair of snaps to which a neuromodulation controller/stimulator may be coupled. The adhesive electrode pad may be configured as an adapter to adapt a forehead/temple non-invasive neuromodulation controller/stimulator apparatus for use on the neck as described herein for treatment of psoriasis.

FIG. 4B shows an example of an adhesive electrode (adapter) of FIG. 4A with a non-invasive neuromodulation controller/stimulator coupled thereto.

FIGS. 4C-4E show front, back and side views, respectively, of an example of a neck-only electrode pad that may be used with a system or apparatus to treat psoriasis, as described herein.

FIG. 5 shows components of a portable, wired non-invasive neuromodulation neurostimulator system.

FIG. 6 shows components of a non-invasive neuromodulation neurostimulator system that connects wirelessly to a control unit comprising a microprocessor.

FIG. 7 shows a workflow for configuring, actuating, and ending a neuromodulation session.

FIGS. 8A-8C illustrate locations for electrode placement of a neck-work neuromodulation controller/stimulator as described herein. The electrodes may be separated by an approximately 1 inch minimum distance and arranged in an anterior to posterior (e.g. foot to head) longitudinal direction, so that the electrodes are stacked atop each other relative in the longitudinal axis. For example, in FIG. 8A, the first (upper) electrode is on the skin over the C1 to C6 regions of the spine, and the second (lower) electrode is over the C2 to C7 region of the spine. In FIG. 8B the first (upper) electrode is in the cervical region of the spine, while the second (lower) electrode is over the thoracic region (e.g., T1 or T2 region) of the spine. In FIG. 8C the distance between the upper and lower electrodes has been increased, but the first (upper) electrode is still in the cervical region while the second (lower) electrode is over the thoracic region.

FIGS. 8D-8F illustrate another example of a neck-worn neuromodulation controller stimulator as described herein. This apparatus may be configured for treatment of an inflammatory disorder, including an inflammatory skin disorder such as psoriasis. FIG. 8D shows a right side view, FIG. 8E shows a back perspective view, and FIG. 8F shows a front perspective view.

FIG. 9A is a table with waveform parameters of another example of a “high F” ensemble waveform as described herein.

FIG. 9B is a table with another variation of an ensemble waveform similar to that shown in FIG. 9A.

FIG. 9C is a table with another variation of an ensemble waveform as shown in FIGS. 9A-9B.

FIG. 9D is a table showing another variation of an ensemble waveform that may be used, e.g., to treat psoriasis.

FIG. 10 is a table showing another example of an ensemble waveform that may be adapted for use as a psoriasis-treating neuromodulation waveform. This variation is consistent with the low F ensemble waveform described herein.

FIG. 11 is a table illustrating one example of a very low F ensemble waveform as described herein.

FIG. 12 is a schematic illustration of a method of treating a patient having psoriasis. Dashed boxes represent optional steps.

FIG. 13 is a chart showing the percentage of users reporting a reducing in stress/anxiety and/or improvement in sleep using a neurostimulator as described herein. The data illustrates the results of a survey of 89 users who previously reported anxiety or problems sleeping (e.g., sleeping <5 hours on average, per night); the users reported an average of 12 sessions per user, average of 16 minutes per use (4 weeks, total of 1108 sessions). Survey asked “have you slept better or had lower stress/anxiety as a result of using [the neurostimulator]”.

FIG. 14 is an illustration of one possible mechanism of action for the use of the apparatuses and methods described herein to treat inflammatory skin disorders such as psoriasis. This proposed mechanism of action is speculative, and not intended to limit the inventions described herein.

FIGS. 15A and 15B illustrate before and after images showing an improvement (typical) in psoriasis in a female subject (between 25-50 years old) with mild psoriasis not using any other medications, following three weeks (30 sessions). FIG. 15A shows an image from the subject's hand showing a mild psoriasis lesion; by week 3 the lesion is gone; overall, the subject reports a reduction or elimination of all lesions in this time period and an overall reduction in itching.

FIGS. 16A-16B illustrate before and after images of a second female subject (25-50 years old) showing improvement in moderate psoriasis over three weeks of use of the methods and apparatuses described herein. FIG. 16A shows a lesion before therapy, behind the subject's ear. FIG. 16B shows the same body region following 3 weeks (12 sessions); the lesion has been reduced/resolved following 3 weeks of treatment.

FIGS. 17A-17B illustrate before and after images of a third female subject (25-50 years old) showing improvement in moderate psoriasis over three weeks of use of the methods and apparatuses described herein. FIG. 17A shows a lesion on the subject's arm before therapy.

FIG. 17B shows the same arm following 3 weeks (29 sessions) of therapy; the lesion has been reduced/resolved following 3 weeks of treatment.

FIG. 18 is a table showing preliminary data from an initial human trial for the treatment of psoriasis, indicating the number of weeks of treatment, the number of sessions, a qualitative description of the extent (if any) of improvement, and self-reported description (diary) from the user.

FIG. 19 is a bar graph showing the effect of a treatment regime as described herein after 4 weeks for treatment and control groups. On the left is the percentage of patients showing at least 50% improvement; on the right is the percent of patients showing at least 75% improvement.

FIG. 20 is a scatter plot showing the percent improvement in patients (for the same study as FIG. 19).

FIG. 21 indicates which patients had severe, moderate, mild for treatment and control patients from FIG. 20.

FIG. 22 is a table of the patient data from FIGS. 19-21.

FIG. 23 is a time course of treatment for a single patient before treatment, at week 2, week 4 and week 5.

FIG. 24 is a diagram illustrating one general concept for the selection of stimulation parameters that may be used.

FIG. 25 is an example of a single waveform cycle for a applying treatment energy, illustrate the positive pulse, open-circuit, negative pulse, and short-circuit (discharge) regions of each “bipolar” pulse used.

FIG. 26 illustrates how each pulse may be combined to form an envelope of pulses.

FIG. 27 illustrates one example of an amplitude-modulated burst of pulses.

FIG. 28 shows relative effects of different waveforms (e.g., in this variation, at different high-frequency components, sham, 7 Khz, variable high frequency, high frequency with amplitude modulation and 500 Hz stimulation.

FIG. 29 is an example of a therapeutic waveform for psoriasis.

FIG. 30 is an example of a ‘sham’ (non-effective) waveform for psoriasis.

FIGS. 31A-31C is an example of a neurostimulator that may be used to treat, e.g., psoriasis, as described herein. FIG. 31A shows a front view of the device (similar to that shown in FIGS. 3A-3F); FIG. 31B shows a back view. FIG. 31C is an example of a gel pad (electrode) that may be electrically and mechanically coupled to the neurostimulator to apply electrical stimulation (e.g., neuromodulation) to the back of the user's neck to treat psoriasis.

FIGS. 32A-32C illustrate the use of neckband that may be worn with the device attached to the back of the user's neck. FIG. 32A shows a neckband portion of the apparatus (“platform”) for positioning and wearing a neurostimulator on the user neck. FIG. 32B shows attachment of the neurostimulator to the neckband to facilitate attachment to a gel pad. FIG. 32C shows the neckband with the neurostimulator attached.

FIGS. 33A and 33B illustrate attachment of the gel pad (electrodes) to the neck band shown in FIGS. 32A-32C.

FIGS. 34A and 34B illustrate wearing of the neurostimulator on the user's neck to treat psoriasis.

FIGS. 35A and 35B are examples of user interfaces for controlling application of therapy by the neurostimulator.

FIG. 36 is an example of a table showing charge per DC phase (in microcoulombs/phase) for a variety of different waveform parameters that may be used to treat psoriasis as described herein.

### DESCRIPTION OF THE INVENTION

In general, described herein are methods and apparatuses (devices and systems) for non-invasively applying electrical energy to treat a medical disorder, including inflammatory (e.g., autoimmune) disorders, skin disorders, and migraines. For example, the methods and apparatuses described herein may be used to treat an inflammatory (and/or autoimmune) disorders such as: rheumatoid arthritis, inflammatory bowel disease, multiple sclerosis, Sjogren's syndrome, Graves' or Hashimoto's thyroiditis, asthma and/or lupus. Other skin-specific disorders that may be treated include, but are not limited to: Pruritus (Itch), Hyper-hidrosis (excessive sweating), facial erythema (facial flush), atopic dermatitis, eczema, prurigo nodularis, lichen planus, chronic urticarial, alopecia areata, rosacea and/or vitiligo. Other medical disorders may include migraines. Although the examples described herein focus primarily on psoriasis, the methods and apparatuses described herein may be used to treat any of the disorders discussed above.

As will be described in greater detail below, particular ranges of stimulation parameters (frequency, peak current amplitude, duty cycle) of non-invasive electrical energy (e.g., neuromodulation) waveforms applied using a wearable electrical energy (e.g., neuromodulation) applicator worn on the subject's neck (or neck and head) have been found to be effective, while stimulation outside of these parameters, and/or at different locations, may not be as effective. For example, stimulation at greater than 10% duty cycle (e.g., between 10 and 90%, between 20 and 80%, between 30 and 80%, etc.), at a frequency that is 100 Hz or greater (e.g., 150 Hz or greater, 200 Hz or greater, 250 Hz or greater, 300 Hz or greater, 400 Hz or greater, 500 Hz or greater, 600 Hz or greater, 700 Hz or greater, 750 Hz or greater, 800 Hz or greater, 1 kHz or greater, 2 kHz or greater, 5 kHz or greater, etc., and in particular, 250 Hz or greater), and a peak amplitude of 3 mA or greater (e.g., 4 mA or greater, 5 mA or greater, 6 mA or greater, 7 mA or greater, 8 mA or greater, 9 mA or greater, 10 mA or greater, etc.) may be particularly effective.

The non-invasively applied neuromodulation waveform may be biphasic and in some variations asymmetric, with respect to positive and negative going phases. In some variations a capacitive discharge (e.g., a rapid depolarization component to discharge capacitance built up on the electrodes (and in the body) may be applied during the pulsed application (e.g., on each or a subset, e.g., during positive going pulses, negative pulses, etc., of the non-invasive neuromodulation stimulation)).

Particular types of non-invasive neuromodulation waveforms delivered to a subject (e.g., to the neck) may enhance the treatment of psoriasis. For example, 15 minute non-invasive neuromodulation waveforms delivered through a wearable non-invasive neuromodulation applicator attached with an anode at the forehead/temple area and cathode on the neck of a subject (delivering a pulsed waveform with variable frequency, generally between 250 Hz and 11 kHz at between 2-12 mA peak current in asymmetric, biphasic pulses) daily in a subject suffering from psoriasis was found to significantly improve the subject's psoriasis, resulting in a reduction in size and number of plaques (e.g., maculopapules).

Described herein are methods and apparatuses for non-invasive neuromodulation electrical stimulation (e.g., neuro stimulation) using non-invasive neuromodulation stimulation protocols and electrode configurations that treat (reduce the number and/or size of plaques/maculopapules) in a subject suffering from psoriasis. Apparatuses described herein may generally include a neurostimulator for delivering non-invasive electrical stimulation, appropriate dermal electrodes that connect electrically to the neurostimulator for transmitting the electrical stimulation to the subject, and, optionally, a controller unit that may be connected to the neuromodulator (e.g., neurostimulator) in a wired or wireless manner (including user computing devices such as a smartphone, tablet, wearable device (e.g. smartwatch or Google Glass), or computer). The non-invasive neuromodulation apparatuses for treating psoriasis described herein may be configured to deliver appropriate neuromodulation waveforms and to couple electrodes with an appropriate configuration.

Any of these methods may include regular (e.g., daily) treatments for a minimum amount of time (e.g., a minimum amount of time having a detectable reduction in sympathetic tone). The apparatus may be adapted to include input from one or more sensors configured or adapted to modify the applied waveform/signal to ensure that the subject is experiencing a minimum duration during which the sympathetic tone is suppresses/decreased. For example, the apparatus may include logic in the controller (or in wireless communication with the controller) to receive and determine from one or more sensors (e.g., heart rate sensors, skin conductance sensors, ECG sensors, EEG sensors, pulse oxygenation, etc.) that the sympathetic tone is decreased and/or parasympathetic tone is decreased.

Any of the methods and apparatuses described herein may be used in conjunction with a medicament (e.g., pharmaceutical agent). For example, when treating psoriasis, the methods may be used in conjunction with a medicament for treating psoriasis or the symptoms of psoriasis and my increase or improve the effectiveness of medicament alone. For example, the methods or apparatuses described herein may be used in conjunction with one or more topical or systemic treatments for psoriasis. Such topical treatments may include one or more of: DOVONEX (calcipotriene), TACLONEX (calcipotriene and betamethasone dipropionate), TAZOREC (tazarotene), VECTICAL (calcitriol), ZITHRANOL-RR (anthralin), coal tar (coal tar extracts), salicylic acid, lactic acid, urea or phenol, etc. Systemic drugs may include one or more of: CIMZIA (Certolizumab pegol), COSENTYX (Secukinumab), ENBREL (Etanercept), HUMIRA (Adalimumab), REMICADE (Infliximab), SIMPONI (Golimumab), STELARA (Ustekinumab), TALTZ (Ixekizumab), Cyclosporine, Methotrexate, OTEZLA (Apremilast), SORIATENE (Acitretin). Topical steroid treatments may include one or more of: Alclometasone dipropionate, Betamethasone dipropionate, Betamethasone valerate, Clobetasol propionate, Desonide, Desoximetasone, Diflorasone diacetate, Fluocinolone acetonide, Fluocinonide, Flurandrenolide, Fluticasone propionate, Halcinonide, Halobetasol propionate, Hydrocortisone, Hydrocortisone valerate, Mometasone furoate, Prednicarbate, and Triamcinolone acetonide.

These neurostimulators may be capable of autonomous function and/or controllable via a wired or wireless connection to a computerized user device (e.g., smartphone, tablet, laptop, other wearable device). The neurostimulator may be configured specifically to deliver stimulation within a range of parameters, including intensity and frequency, determined to be effective for treating psoriasis while minimizing pain and discomfort due to the relatively large magnitude stimulation provided. For example, an apparatus (such as a non-invasive ANS neuromodulation applicator) may include a control module having circuitry (e.g., hardware), software and/or firmware that allows the apparatus to apply signals within an effective range, including, for example, one or more processors, timers, and waveform generators.

These apparatuses may use replaceable, disposable (e.g., consumable) electrodes and may also use appropriate electrical stimulation parameters; this combination may mitigate discomfort, enabling higher peak currents to be delivered for stimulating transdermally without delivering irritating or painful stimuli that may wake a subject. Higher peak currents typically provide a more robust effect.

A neurostimulation system as described herein may include two or more parts: (1) a lightweight (e.g., less than 100 g, less than 75 g, less than 50 g, less than 40 g, less than 30 g, less than 25 g, less than 20 g, etc.), wearable (or portable), neurostimulator device (neurostimulator) that is configured to be worn on a subject (generally on the head or neck) or portable and coupled to the subject and includes processor(s) and/or controller(s) to prepare the non-invasive neuromodulation waveform(s) to be applied; and (2) a consumable/disposable electrode assembly to deliver the non-invasive neuromodulation waveform(s) to the wearer. In some variations a third component may be a controller that is separate from but communicates with the neurostimulator. For example, in some variations the controller may be a user device that wirelessly communicates with the neurostimulator. In some variations the controller is a mobile telecommunications device (e.g., smartphone or tablet) being controlled by an application that sends instructions and exchanges 2-way communication signals with the neurostimulator. For example, the controller may be software, hardware, or firmware, and may include an application that can be downloaded by the user to run on a wireless-connectable (e.g., by Bluetooth) device (e.g., smartphone or tablet) to allow the user to select the waveforms delivered by the neurostimulator, including allowing real-time or short latency (e.g., less than one second, less than 500 ms, etc.) modulation of the delivered neuro stimulation to treat psoriasis as described herein. Alternatively, the electrodes may be reusable and integrated in a single assembly with a non-invasive neuromodulation controller.

The methods and apparatuses described herein may induce a calm or relaxed mental state (e.g., during which the sympathetic tone is decreased) and may facilitate, induce, or maintain this state for greater than a predetermined period (e.g., greater than 5 minutes, 10 minutes, 15 minutes, 20 minutes, etc.) during a treatment session. This class of cognitive effects includes those associated with relaxation and a calm mental state, for example: a state of calm, including states of calm that can be rapidly induced (e.g., within about 5 minutes of starting delivery of the non-invasive neuromodulation waveforms). In some variations, these effects may include a reduction in psychophysiological arousal as associated with changes in the activity of the hypothalamic-pituitary-adrenal axis (HPA axis) and/or reticular activating system and/or by modulating the balance of activity between the sympathetic and parasympathetic nervous systems generally associated with a reduction in biomarkers of stress, anxiety, and mental dysfunction; anxiolysis; a state of high mental clarity; enhanced physical performance; promotion of resilience to the deleterious consequences of stress; a physical sensation of relaxation in the periphery (i.e. arms and/or legs); a physical sensation of being able to hear your heart beating, and the like.

The apparatuses (systems and devices) and methods described herein allow the reproducible reduction in skin effects (e.g., plaques) associated with psoriasis. The effect resulting from the methods and devices described may depend, at least in part, on the positioning of the electrodes. It may be particularly advantageous with the non-invasive neuromodulation waveform parameters described herein to apply the electrodes on the subject's neck, neck and head, or neck and elsewhere on the body other than the head. Described below are three configurations for treating psoriasis. These configurations are exemplary and are not meant to be limiting with regard to configurations that can induce these cognitive effects and thus treat psoriasis in a subject.

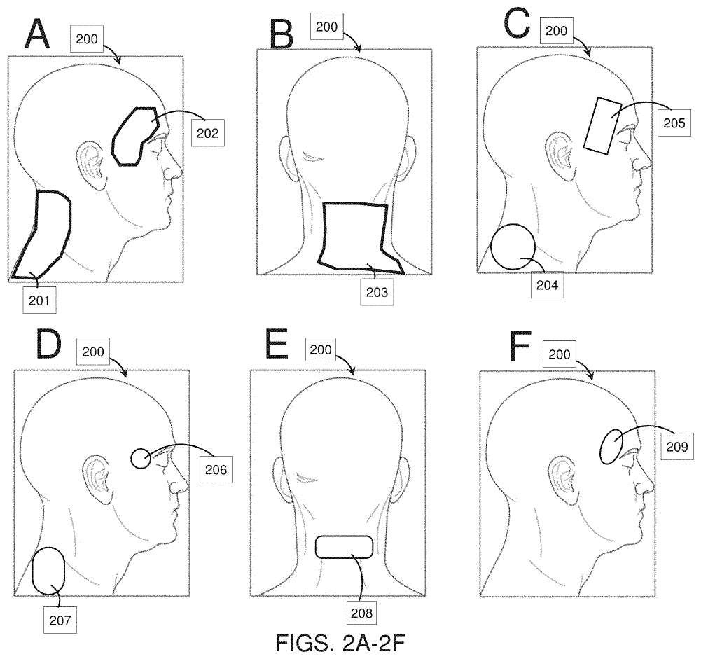

FIGS. 2A-2F illustrate electrode configurations that may be used for treating psoriasis in a subject 200 and may be referred to herein. Typically, these configurations include at least one electrode on the subject's neck and a second electrode that may also be placed on the subject's neck and/or shoulder, mastoid region, or head (e.g., temple). For example, a first electrode may be positioned on the subject's skin near the subject's neck (e.g., on a superior portion of the neck center as in FIG. 2E). Beneficial embodiments comprise electrodes for the neck having an area of at least about 20 cm². In one example, an electrode having area at least about 10 cm² (optimally at least about 20 cm²) is placed near the right temple. FIGS. 2A and 2B show the broad outlines of effective areas for a neck 201, 203 electrode with a temple electrode 202 (though the actual electrodes within these areas may be smaller than the regions outlined). For example, effective electrode size and positions may be as shown in FIG. 2C, wherein rectangular temple electrode 205 and circular electrode (on the right side of the neck) 204 are applied to the subject. In another example of effective electrode size and positions shown in FIG. 2D, a small circular temple electrode 206 and elongated oval electrode (on the right side of the neck) 207 are applied to the subject. In a third example of effective electrode size and positions shown in FIGS. 2E-2F, an oval temple electrode 209 and roughly rectangular electrode (centered on the superior portion of the neck) 208 are applied to the subject.

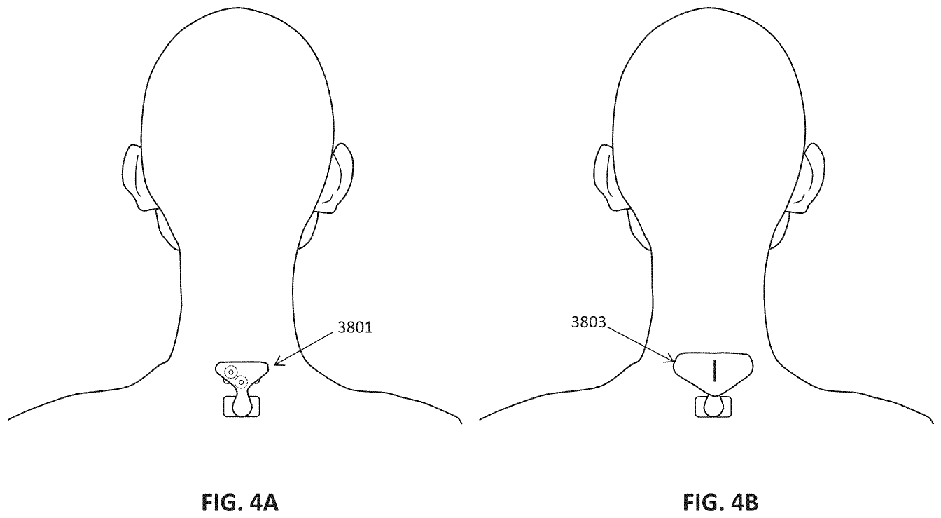

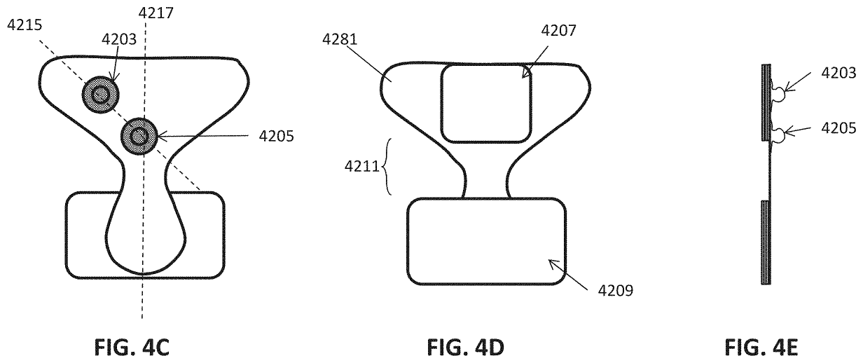

FIGS. 4A-4B illustrate a second electrode configuration for treating psoriasis in subject. In this example, FIGS. 4A-4B illustrate the use of an electrode pad 3801 (also referred to herein as a neck-only electrode pad) that is configured to be worn over the C3-T2 spinal region on the skin, in which the closest-edge to closest-edge separation between the first and second electrode of the pair of electrodes is separated by between 0.8 inches and 2.5 inches (e.g., 0.8 inches and 1.6 inches). In this example, the adapter electrode pad 3801 is placed on the skin over the C3-T2 region of the spine, so that the electrodes are arranged in the midline of the back/neck in the longitudinal anterior-to-posterior axis, with the lower electrode over the C5-T2 region. This is shown in FIG. 4A. In this example, the adapter electrode pad includes a pair of male connectors, shown configured as snaps having protrusions which mate with female connectors on a non-invasive neuromodulation controller device 3803, providing mechanical and electrical connection. The non-invasive neuromodulation controlling device may be a lightweight wearable non-invasive neuromodulation controller device, including those incorporated for reference above, which are otherwise configured to be worn on the subject's head. The adapter electrode pad is therefore configured to adapt these device so that they can be worn on the neck, as shown in FIG. 4B.d

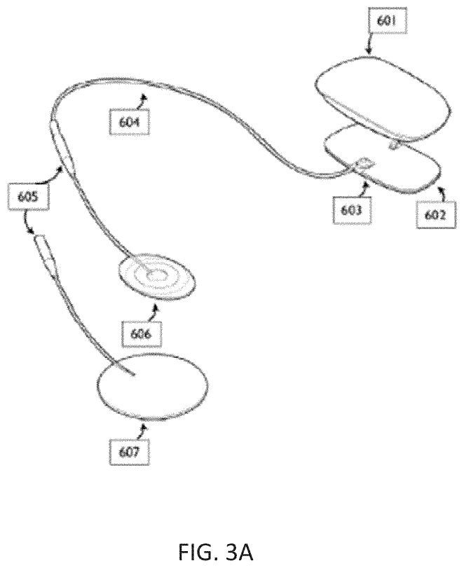

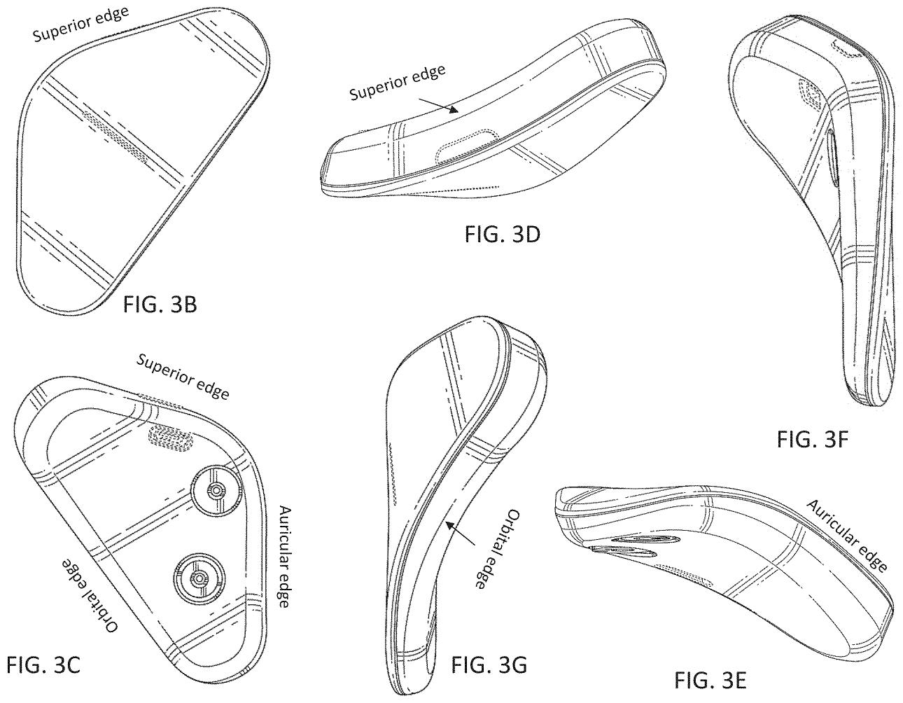

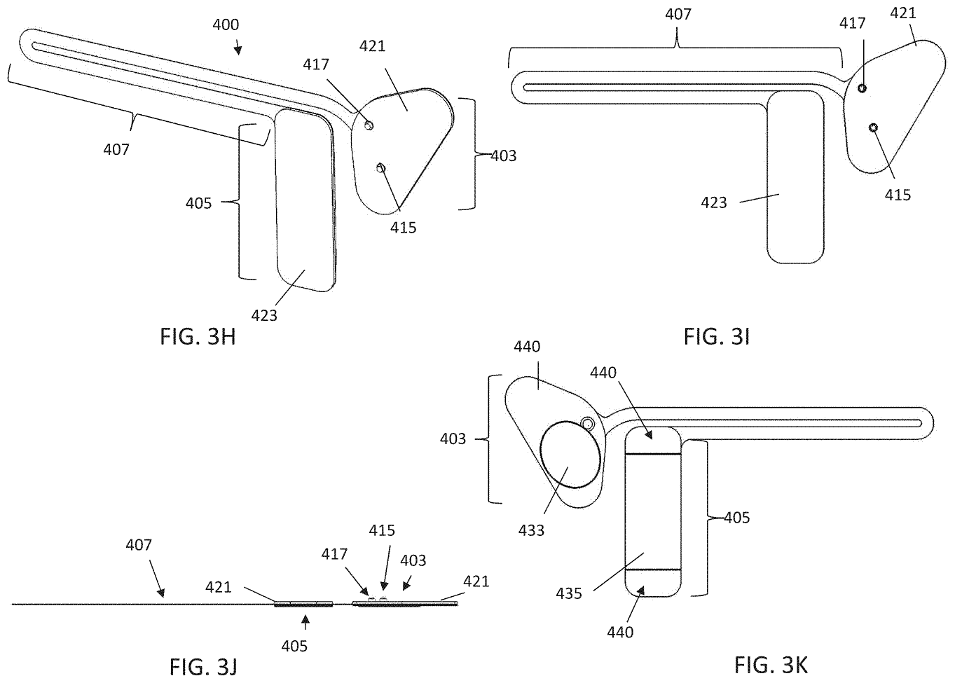

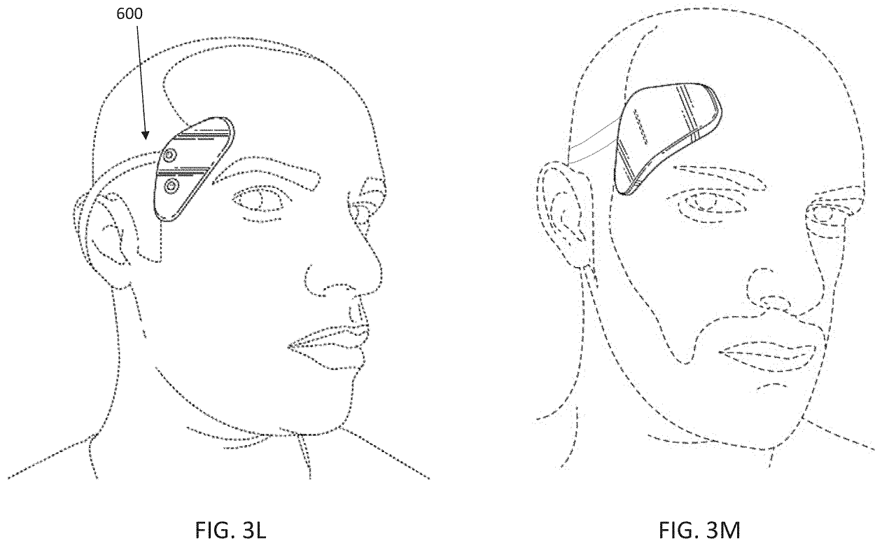

FIGS. 4C-4E illustrate an example of a neck-only electrode pads that may be used. In FIG. 4C, the adapter electrode pad includes a pair of connectors 4203,4205 that are shown as male snap type connectors that may make an mechanical and electrical connection with the non-invasive neuromodulation controller device, as shown in FIG. 3B-3F. The electrode pad is generally flat, and is configured so that it can be flexible, yet provide good contact between an upper electrode 4207 and the skin and a lower electrode 4209. As shown in FIG. 4D, the upper electrode may be separated from the lower electrode (closet edge to closet edge 4211) by between about 0.8 inches and 2.5 inches. In FIG. 4D the distance is approximately 1 inch.

The electrode pad shown in FIGS. 4C-4E are configured for applying non-invasive neuromodulation to the back of a subject's neck. Any of these electrode pads may include a flat substrate 4281; the first (e.g. upper) electrode 4207 on a first side of the flat substrate and a second (e.g., lower) electrode 4209 also on the first side. As mentioned, the closest edge of the first electrode is separated from a closest edge of the second electrode by between 0.8 inches and 2 inches 4211. These electrode pads may also include a first male snap connector 4203 that is electrically connected to the first electrode and extends from the substrate on a second side of the flat substrate that is opposite from the first side. A second male snap connector 4205 electrically connects to the second electrode and extends from the substrate on the second side.

In any of these variations, the electrode pad may be adhesively held to the skin. For example, the first side may comprise an adhesive. As mentioned, the flat substrate may have a two-lobed (e.g., bi-lobed) shape. The first electrode and the first and second male snap connectors may be on a first lobe of the flat substrate and wherein the second electrode may be on a second lobe of the flat substrate, as shown in FIGS. 4C-4E. The second electrode may extend beyond the perimeter of the flat substrate, as shown. In general, the second electrode may be larger than the first electrode. For example, the surface area of the second electrode may be greater than 1.25 times (e.g., greater than 1.4×, greater than 1.5×, greater than 1.6×, greater than 1.7×, greater than 1.8×, greater than 1.9×, greater than 2×, etc.) the surface area of the first electrode. As mentioned, the closest edge of the first electrode may be separated from the closest edge of the second electrode by between 0.9 and 1.5 inches, preferably around 1 inch.

In this example, the electrode pad is formed from a flexible substrate onto which each electrode is formed by adding layers, as illustrated schematically in FIG. 4F.

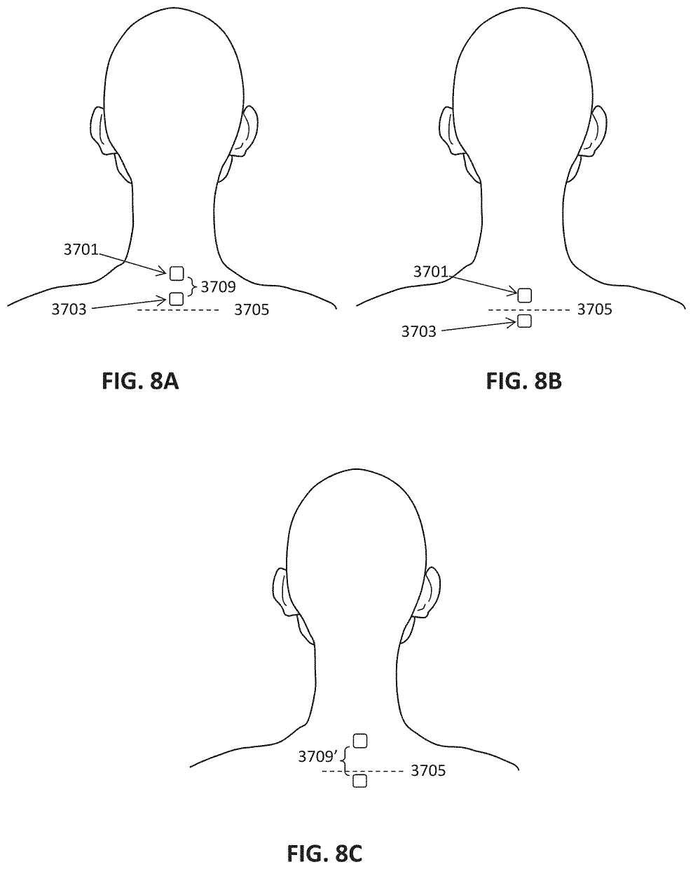

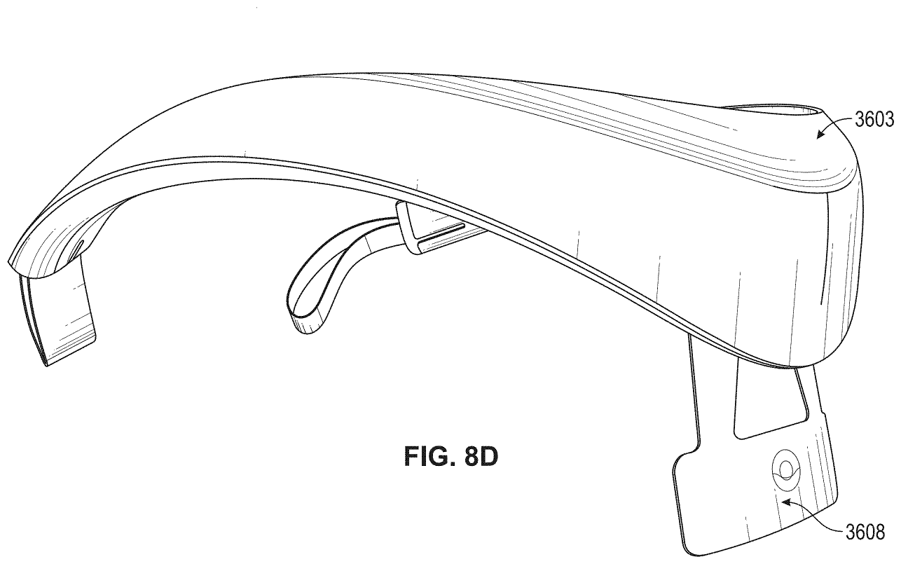

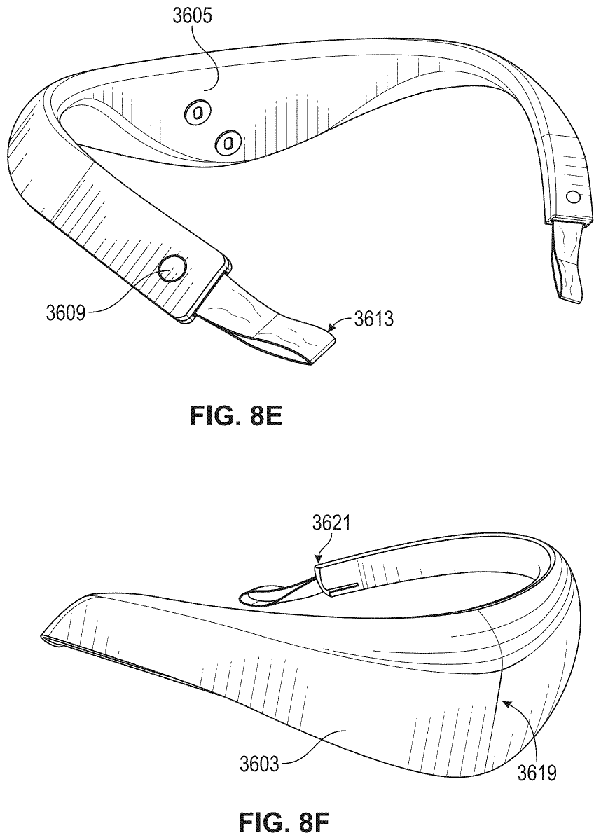

FIGS. 8A-8C illustrate electrode configurations that may be used to treat psoriasis or other inflammatory disorders (including inflammatory skin disorders). In FIG. 8A-8C, the lower electrode may be positioned on the skin over the upper thoracic region of the spine; the upper electrode may also be positioned over the upper thoracic region or in the lower cervical region. For example. FIGS. 8A-8C illustrate variations with this positioning. In FIG. 8A, the pair of electrodes includes a first electrode 3701 in which the upper electrode is within the lower cervical region (e.g., on the skin over the C4-C6 region of the spine), while the second electrode 3703 is also over the lower cervical region of the spine (e.g., on the skin over the C3-C7 region of the spine). More preferably, as shown in FIG. 8B, the upper electrode 3701 is positioned over the lower cervical region (e.g., C6-C7) while the lower electrode 3703 is positioned at the top of the thoracic region (e.g., T1-T2). In FIGS. 8A-8B, the division between the cervical and thoracic region is approximately shown by dashed line 3705. The upper and lower electrodes may be part of an electrode pad that is separate from or integral with the non-invasive neuromodulation controller device.

In general, in any of the methods and apparatuses described herein, it may be beneficial for the electrodes to be arranged so that the first electrode is above the second electrode when worn on the body along the subject's anterior-to-posterior (e.g. foot-to-head) longitudinal midline at the back of the neck/upper back. The separation between the first and second electrodes may also be important. For example, the separation may be between 0.7 inches and 2 inches, preferably between 0.8 inches and 1.4 inches. The minimum distance may be between 0.7 and 1.2 inches (e.g., approximately 1 inch), from the nearest edge to the nearest edge. The maximum distance may be between 1.7 inches and 2.2 inches (e.g., 2 inches) from nearest edge to nearest edge. For example, as shown in FIG. 8A, the electrodes may be separated 3709 by an approximately 0.8-1.5 inch distance (nearest edge to nearest edge) and arranged in an anterior to posterior (e.g. foot to head) longitudinal direction, so that the electrodes are stacked atop each other relative in the longitudinal axis.

FIG. 8C illustrates an example of an arrangement of the electrodes in which the upper electrode is on the skin over the cervical region while the lower electrode is on the skin over the thoracic region of the spine, similar to FIG. 8B, however the separation 3709′ between the electrodes (nearest edge to nearest edge) is closer to 2 inches (e.g., between 1.8 and 2.2 inches). In general, the minimum distance between the electrodes may provide field penetration of sufficient depth so that the energy is not simply shunted across the subject's skin. Without being bound to a particular theory of operation, this may allow stimulation of the cervical nerves. However, if the electrodes are too far apart, the energy applied may be too diffuse or may require a larger output energy. Surprisingly, having the electrodes separated by approximately 1 inch (nearest edge to nearest edge) works, and indeed works particularly well.

FIGS. 8D-8F illustrate another example of a neck-worn device that may be used to treat psoriasis. In this example, the non-invasive neuromodulation apparatus includes a rigid or semi-rigid frame 3603. In some variations the frame may be formed of a polymeric material, such as a plastic material, including metallized plastics. The inner surface of the frame may be padded, covered, coated, etc. for wearing comfort. For example, the inner (user-facing) surface may be wrapped or covered with a fabric 3605. One or more electrodes, or attachments/connectors for a disposable electrode (e.g., strip, pad, contact strip, etc.) may be present on the inner surface as shown in FIG. 8E, or it may be present inside of the surface, or on an outer surface, and the pad may extend down/up from the wearable body. FIG. 8D illustrates an example of an electrode strip/pad 3608 extending from the wearable body. The strip or pad may be snapped or otherwise coupled to the wearable body. In FIG. 8E the inner surface of the body shows a pair of offset connectors for coupling (in this example, snap-fitting) to the pad. The electrodes 3608 may be held against the skin (e.g., adhesively or simply by virtue of the connection to the weight of the wearable body). The body 3603 may also be textured on the outer, inner, or both surfaces (e.g., an in-mold texture on plastic in this example). In some variations the connections to the electrodes may be present within the housing 3603, which may include a slot, clamp, or the like to hold the electrode connectors and make connection thereto. Alternatively, as described above, the electrodes may be reusable, durable electrodes that are coupled to and/or extend from the wearable body.

In FIG. 8E the wearable body also includes at least one control (e.g., power button 3609) on the body. Additional controls (buttons, sliders, switches, etc.) may be included; alternatively no buttons may be present on the surface, but it may be powered on/off remotely and/or controlled remotely, e.g., by a wireless apparatus such as a smartphone running control software.

The apparatus of FIGS. 8D-8F includes one or more straps 3613 (e.g., nylon straps 3611) that may be present at the ends of the torque-shaped neck worn body and may be used to attach to an additional component (e.g., leash, etc.) or may be configured to attach to clothing or jewelry. The ends of the arms of the wearable body may be metallic (e.g., may include metallic endcaps 3621, as shown in FIG. 8F). The wearable body may also include one or more indicator light regions 3619 which may be illuminated by one or more (including different color, intensity, etc.) light sources, such as LEDs.

Additional electrode configurations for treating psoriasis may include: a first electrode on the neck and a second electrode on the shoulder (i.e., deltoid, upper arm, etc.); one electrode on each shoulder (i.e., deltoid, upper arm, etc.).

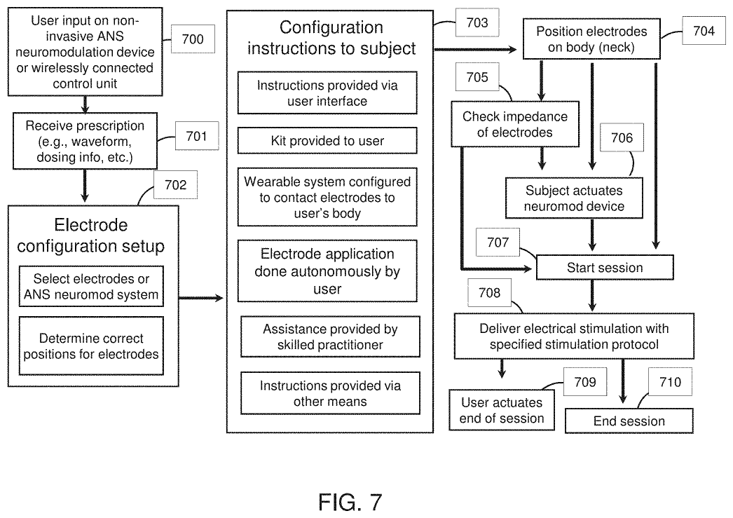

FIG. 7 shows an exemplary workflow for configuring, actuating, and ending a non-invasive neuromodulation session for treating psoriasis. According to an embodiment of the present invention, user input on non-invasive neuromodulation device or wirelessly connected control unit 700 is used to select desired cognitive effect 701 which determines electrode configuration setup 702 to achieve the desired cognitive effect, including selection of electrodes or a non-invasive neuromodulation system that contains electrodes and determination of correct positions for electrodes. As described above, these configurations may be beneficial for treating psoriasis. Neck-specific, including neck-only, configurations may be particularly beneficial.

Configuration instructions to a user 703 may be provided by one or more ways selected from the list including but not limited to: instructions provided via user interface; kit provided to user; wearable system configured to contact non-invasive neuromodulation electrodes to appropriate portions of a user's body; and instructions provided via other means.

Based on these instructions or knowledge, a patient (or a user working with the patient) or other individual or system positions electrodes on body 704. In some embodiments, the non-invasive neuromodulation session starts 707 automatically after electrodes are positioned on the body. In other embodiments, the impedance of the electrodes 705 is checked by a non-invasive neuromodulation system before the non-invasive neuromodulation session starts 707. In some embodiments, after impedance of the electrodes 705 is checked by a non-invasive neuromodulation system, user actuates non-invasive neuromodulation device 706 before the non-invasive neuromodulation session starts 707. In other embodiments, after positioning electrodes on the body 704 the user actuates the non-invasive neuromodulation device 706 to start the non-invasive neuromodulation session 707. Once the non-invasive neuromodulation session starts, the next step is to deliver electrical stimulation with specified stimulation protocol 708. In some embodiments, a user actuates end of non-invasive neuromodulation session 709. In other embodiments, the non-invasive neuromodulation session ends automatically when the stimulation protocol completes 710.

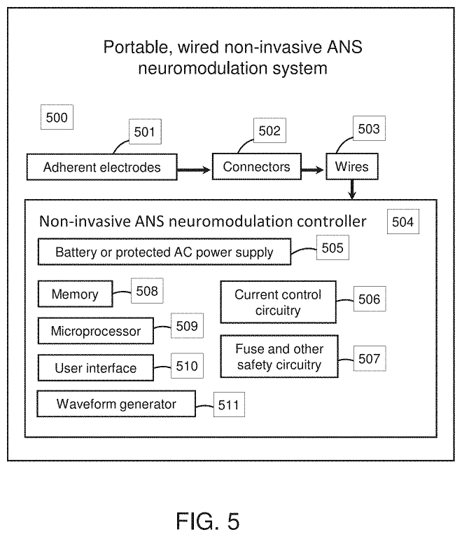

FIG. 5 shows a schematic illustration of a portable, wired non-invasive neuromodulation neurostimulator 500. According to an embodiment, adherent electrodes 501 connect to non-invasive neuromodulation controller 504 via connectors 502 and wires 503. Non-invasive neuromodulation controller 504 has several components including battery or protected AC power supply 505, fuse and other safety circuitry 507, memory 508, microprocessor 509, user interface 510, current control circuitry 506, and waveform generator 511.

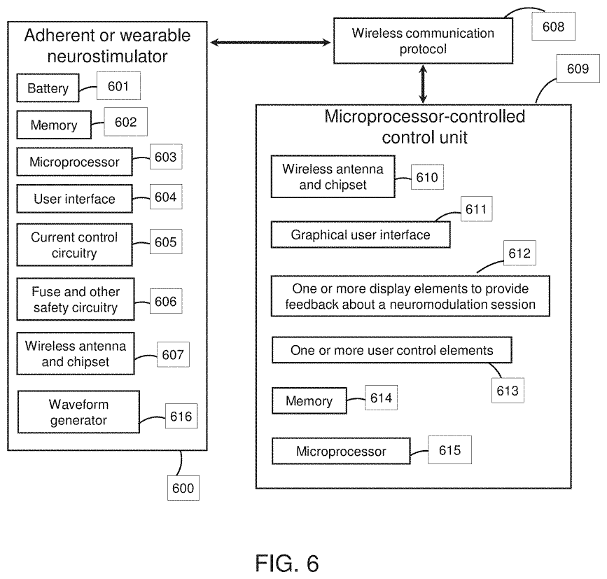

FIG. 6 shows an embodiment of a non-invasive neuromodulation system comprising adherent or wearable non-invasive neuromodulation neurostimulator 600 that communicates wirelessly with microprocessor-controlled control unit 609 (e.g., a smartphone running an Android or iOS operating system such as an iPhone or Samsung Galaxy, a tablet such as an iPad, a personal computer including, but not limited to, laptops and desktop computers, or any other suitable computing device). In this exemplary embodiment, adherent or wearable neurostimulator 600 holds two or more electrodes in dermal contact with a subject with one or more of: an adhesive, a shaped form factor that fits on or is worn on a portion of a user's body (e.g., a headband or around-the-ear ‘eyeglass’ style form factor). In an exemplar embodiment, adherent or wearable neurostimulator 600 comprises components: battery 601, memory 602, microprocessor 603, user interface 604, current control circuitry 605, fuse and other safety circuitry 606, wireless antenna and chipset 607, and waveform generator 616. Microprocessor-controlled control unit 609 includes components: wireless antenna and chipset 610, graphical user interface 611, one or more display elements to provide feedback about a non-invasive neuromodulation session 612, one or more user control elements 613, memory 614, and microprocessor 66. In an alternate embodiment the neurostimulator 600 may include additional or fewer components. One of ordinary skill in the art would appreciate that neurostimulator could be comprised of a variety of components, and embodiments of the present invention are contemplated for use any such component.

An adherent or wearable neurostimulator 600 may be configured to communicate bidirectionally with wireless communication protocol 608 to microprocessor-controlled system 609. The system can be configured to communicate various forms of data wirelessly, including, but not limited to, trigger signals, control signals, safety alert signals, stimulation timing, stimulation duration, stimulation intensity, other aspects of stimulation protocol, electrode quality, electrode impedance, and battery levels. Communication may be made with devices and controllers using methods known in the art, including but not limited to, RF, Wi-Fi, WiMax, Bluetooth, BLE, UHF, NHF, GSM, CDMA, LAN, WAN, or another wireless protocol. Pulsed infrared light as transmitted for instance by a remote control is an additional wireless form of communication. Near Field Communication (NFC) is another useful technique for communicating with a neuromodulation system or neuromodulation puck. One of ordinary skill in the art would appreciate that there are numerous wireless communication protocols that could be utilized with embodiments of the present invention, and embodiments of the present invention are contemplated for use with any wireless communication protocol.

Adherent or wearable neurostimulators 609 may or may not include a user interface 604 and may be controlled exclusively through wireless communication protocol 608 to control unit 609. In an alternate embodiment, adherent or wearable neurostimulator 609 does not include wireless antenna and chipset 607 and is controlled exclusively through user interface 604. One skilled in the art will recognize that alternative neurostimulator systems can be designed with multiple configurations while still being capable of delivering electrical stimulation transdermally into a subject.

In general, any appropriate neurostimulation system may use (and/or be configured to use or operate with) the ensemble waveforms as described herein for treating psoriasis. FIGS. 3A, and 3B-3M describe and illustrate an example of a neurostimulation system (neurostimulator, electrodes, controller) that may be used. For example, a neurostimulation system may include a lightweight, wearable, neurostimulator device (neurostimulator) that is configured to be worn on the head and a consumable/disposable electrode assembly; in addition a device that may be worn and/or held by the user (“user device”) which includes a processor and wireless communication module may be used to control the application of neurostimulation by the wearable neurostimulator. The neurostimulator and/or user device may be particularly adapted to deliver the ensemble waveforms as described herein. For example, the user device may present a list of ensemble waveforms and allow the user to select among them in order to select a desired cognitive effect. The ensemble waveforms may be ordered by the desired effect (e.g., treating psoriasis, including reducing the number and/or size of plaques/maculopapules, etc.) and/or by time and/or by ranking, etc. Further, the user device may be adapted to communicate with the wearable neurostimulator and may transmit an identifier of the selected ensemble waveform, and/or waveform parameters that define all of a portion (e.g., component waveforms or portions of component waveforms) of the ensemble waveform, as well as any user adjustments such as user modification to the perceived intensity to be used to modify the actual waveforms delivered by, for example, attenuating the ensemble waveform parameters. Thus, for example, the user device may be configured to send, and the neurostimulator to receive, the ensemble waveform parameters (duration, ramping parameter/ramping time, capacitive discharge parameters, current amplitude, frequency, percent duty cycle, percent charge imbalance, etc.).

The user device may also be referred to herein as a controller, and the controller (user device or user computing device) is typically separate from but communicates with the neurostimulator. For example, in some variations the controller may be a user device that wirelessly communicates with the neurostimulator. In some variations the controller is a mobile telecommunications device (e.g., smartphone or tablet) or wearable electronics (e.g., Google glass, smart watch, etc.), being controlled by an application that sends instructions and exchanges 2-way communication signals with the neurostimulator. Any of these embodiments may be referred to as handheld devices, as they may be held in a user's hand or worn on the user's person. However, non-handheld control user devices (e.g., desktop computers, etc.) may be used as well. The user device may be a general purpose device (e.g., smartphone) running application software that specifically configures it for use as a controller, or it may be a custom device that is configured specifically (and potentially exclusively) for use with the neurostimulators described herein. For example, the controller may be software, hardware, or firmware, and may include an application that can be downloaded by the user to run on a wireless-connectable (i.e., by Bluetooth) device (e.g., handheld device such as a smartphone or tablet) to allow the user to select the waveforms delivered by the neurostimulator, including allowing real-time modulation of the delivered neurostimulation to modify the user's cognitive state as described herein.

The neurostimulator may apply an ensemble waveform for about 3-30 min (or longer) that is made up of different “blocks” having repeated waveform characteristics; the waveform ensemble may include transition regions between the different blocks. In general, at least some of the waveform blocks (and in some variations most or all of them) generally have a current amplitude of >3 mA (e.g., >3 mA, greater than 4 mA, greater than 5 mA, between 5 mA and 40 mA, between 5 mA and 30 mA, between 5 mA and 22 mA, etc.), and a frequency of >100 Hz (e.g., between 750 Hz and 25 kHz, between 750 Hz and 20 kHz, between 750 Hz and 15 kHz, etc.), the current is typically biphasic and is charge imbalanced, and has a duty cycle of between 1-90% (e.g., between 10-90%, between 30-80%, between 30-60%, etc.). One or more of these characteristics may be changed during stimulation over timescales of every few seconds to minutes as the ensemble waveform shifts between subsequent component waveforms.

When worn, the system may resemble the system shown in FIG. 3M, having an electrode assembly attached at two locations (points or regions) on the subject's head and/or neck) and a neurostimulator attached to the electrode assembly, as shown; in some variations a separate controller may be attached to coordinate the application of stimulation.

As will be described in greater detail herein, the neurostimulator may be lightweight (e.g., less than 30 g, less than 25 g, less than 20 g, less than 18 g, less than 15 g, etc.), and self-contained, e.g. enclosing the circuitry, power supply, and wireless communication components such as a rechargeable battery and charging circuit, Bluetooth chip and antenna, microcontroller, and current source configured to deliver waveforms with a duration of between 10 seconds and tens of minutes. A neurostimulator may also include safety circuitry. The neurostimulator may also include circuits to determine that the electrode is attached and what “kind” of electrode it is (i.e., for configuration 3 vs. configuration 4; or indicating the batch and/or source of manufacture, etc.). FIGS. 3A and 3B-3G illustrate two variations of a neurostimulator.

For example, FIG. 3A illustrates a first example of a neurostimulator as described herein. In FIG. 3A, the neurostimulator is shown with a pair of electrodes attached. A first electrode 601 is coupled directly to the body 603 of the non-invasive neuromodulation applicator 602, and a second electrode 606 is connected by a cable or wire 604 to the body 603 of the applicator 602. These electrodes are separate from each other, and may be replaceable/disposable. Different shaped electrodes 607 may be used with the same re-usable neurostimulator. The neurostimulator in this example includes a rigid outer body, to which the pair of electrodes is attachable, making electrical contact via one or more plug-type connectors.

FIGS. 3B-3G illustrate another embodiment of a neurostimulator as described herein. In this variation the neurostimulator is also a lightweight, wearable neurostimulator that attaches to an electrode, and includes contacts for making an electrical connection with two (or potentially more) electrically active regions (e.g., anodic and cathodic regions) on the electrode(s). However, in this example, the neurostimulator is configured to operate with a cantilevered electrode apparatus, and to attach both mechanically and electrically to the electrode apparatus at a region that is off-center on the bottom (underside or skin-facing side) of the neurostimulator, allowing one end region to be held securely to the skin while the other edge region is not pinned in this way. The “floating” end may therefore adjust slightly to different curvatures of the head, even while the electrode assembly (which may be flexible) is securely held to the skin. Thus, this cantilevered attachment mechanism may enhance comfort and adjustability of the device. In addition, the neurostimulator device may be configured specifically so that it can be comfortably worn at the user's temple, even in users wearing glasses. For example, the apparatus may be configured so that the skin-facing side (which connects to the electrode assembly via one or more connectors) is curved with a slightly concave surface having a slight twist angle. This curve shape may help the apparatus fit more snugly (more uniformly) to the surface of the temple. In addition, one end of the device (the end to be positioned in-line with the edge of the user's eye and the user's ear) may be thinner (e.g., less than 2 cm, less than 1.5 cm, less than 1 cm, less than 0.8 cm, etc.) than the opposite end, which may be worn higher up on the temple.

For example, FIGS. 3B-3G illustrate front, back, left side, right side, top and bottom perspective views, respectively of a variation of a neuro stimulation device (neurostimulator or electrical stimulator) that may be used with cantilever electrode apparatuses. The overall shape of the neurostimulator may be triangular, and particularly the surface of the neurostimulator (though curved/concave and twisted) adapted to connect to the electrode apparatus and face the patient may be three-sided (e.g., roughly triangular). This roughly triangular shape may include rounded edges, and the thickness of the stimulator (in the direction perpendicular to the surface contacting the cantilever electrode apparatus) may vary, e.g., be thinner along one side, and particularly the side (the portion between the orbital edge and the auricular edge) that will extend laterally from the edge of the eye in the direction of the ear. This shape may also be beneficial when helping to fit/be worn on most people in a region of the face/head that tends to not have hair. Both adhesive and conductive hydrogel that may cover an active electrode region function more effectively on skin with little or no hair. This thin lower corner (the orbital/auricular corner) may fit between the eyebrow and hairline, while the wider portion is positioned up in the forehead area where there is less likely to be hair.

In FIGS. 3B-3G the various edges of the neurostimulator are labeled, based on where the apparatus will be worn by the subject, as is illustrated in FIG. 3M. In general, the side of the unit worn toward the ear is the auricular edge, the side worn highest on the forehead is the superior edge, and the side worn nearest the eye/eyebrow is the orbital edge. The overall shape of the neurostimulator is triangular (including rounded edges). As used herein triangular includes shapes having rounded/smooth transitions between the three sides, as illustrated. The subject-facing surface is specifically contoured to fit in the predefined orientation, making it difficult or impossible for a subject to misapply, and risk placing the active region of the attached cantilever electrode apparatus in the wrong place. When attaching the cantilever electrode apparatus to the neurostimulator, the cantilever electrode apparatus may flex or bend so that it is contoured to match the curved and twisted surface. This surface is a section of a saddle shape, in which there is an axis of curvature around which the surface is concavely curved, and an axis of twisting, which may distort the curved surface (the two axes may be different or the same).

Within the housing, any of the neurostimulators described herein may include a processor (e.g., microprocessor) or controller, a wireless communication module that is connected to the processor, and a power source (e.g., battery, etc.). The power source may be configured to provide power to the internal circuitry and/or the circuitry driving current between anodic and cathodic regions of the electrodes when worn by the user. The power supply may be a high-voltage power supply, e.g., able to provide up to 60 V across these electrode terminals. In general, the apparatus may also include circuitry that is configured to regulate the energy (e.g., current) delivered as required by the processor, which may in turn receive instructions via the wireless communications module from a controller. The controller may also communicate information, and in particular information about the electrodes, including confirming that the electrode assembly is connected and/or what type (e.g., calm, energy, make/model, batch, etc.) of electrode assembly is attached, and an indicator of the contact with the user's skin (e.g., conductance, a parameter proportional to conductance, or a value from which an estimate of the conductance of the electrode(s) may be derived).

The electrode assembly may mechanically and/or electrically connect to the neurostimulator, e.g., by snapping to the underside of the neurostimulator at one or more (e.g., two) connectors such as snap receivers. Thus in some variations the neurostimulator may be held onto the subject's (user's) head by the electrode assembly; the electrode assembly may be adhesively connected to the user's head and/or neck to form an electrical contact with the desired regions on the user, and the neurostimulator may be connected e.g., adhesively and/or electrically, to the electrode assembly. As described below, the connectors between the neurostimulator and the electrode assembly may be positioned in a particular and predetermined location that allows the neurostimulator to be robustly connected to the electrode assembly and therefore the user's head/neck without disrupting the connection, and while permitting the system to be worn on a variety of different body shapes.

Electrode assemblies are generally described in detail below, along with specific examples and variations. In particular, described herein are electrode assemblies that are thin (e.g., generally less than 4 mm, less than 3 mm, less than 2 mm, less than 1 mm, etc. thick, which may not include the thickness of the connectors that may extend proud from the thin electrode assembly), and flexible, and may be flat (e.g., formed in a plane). For example, they may be printed on a flex material, such as the material used to print a flex circuit. In use, they can be wrapped around the head to contact it in at least two locations (e.g. at the temple and on the back of the neck). The electrode assembly may include a connector (electrical and/or mechanical) that extends proud of the otherwise flat/planar surface to connect the active regions of the electrode assembly to the neurostimulator. For example, the neurostimulator may be mechanically and electrically connected by one or more snaps extending from the front of the electrode assembly. In some examples, one snap connects to a first active electrode region (anodic or cathodic region) that is surrounded by an adhesive to adhere the active region to the user's head. A second electrode region (anodic or cathodic) on a separate part of the electrode assembly may be electrically connected to the other connector. For example, the second electrode region may be adapted to fit either on a region across the user's neck at the base of the hairline, e.g., near the midline of the neck (calm electrode configuration).

The electrode apparatus may be printed (e.g., by flexographic printing, laser printing with conductive ink, silk-screening, etc.) on a flexible (e.g. plastic) substrate (flex substrate) and may also include a pair of connectors (snaps) on the side opposite the skin-facing electrodes. The electrode active regions on the back of the assembly may include a layer of conductor (e.g., silver), over which a layer of Ag/AgCl is placed that is sacrificial and acts as a pH buffer. A next layer of hydrogel overlays the Ag/AgCl electrode so that it can uniformly transfer charge across the active region into the skin. A portion of the electrode assembly around the active electrode area may have an adhesive that permits good contact with a user's skin.

There may be multiple configurations (e.g., shapes) of the electrode assembly, and, as described in greater detail herein, the electrode assembly may generally be formed on a flexible material (‘flex circuit’ material) and mechanically and electrically connected to the neuro stimulator.

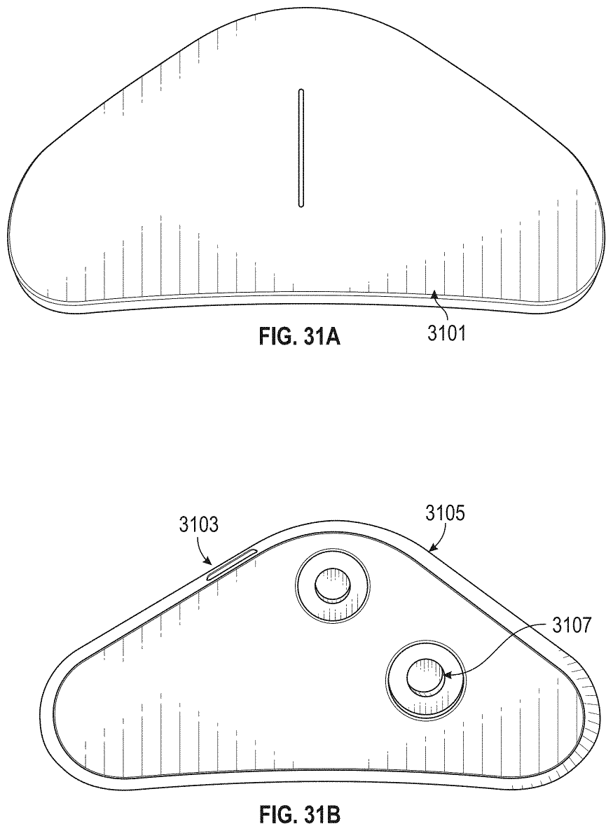

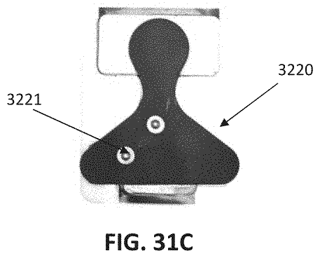

FIGS. 3H-3K illustrate one variation of a cantilever electrode apparatus (“electrode apparatus”) that may be used with a neurostimulator and may be worn on a subject's neck and head. This variation is adapted to connect to a user's temple region and the back of a user's neck. In this example, the cantilever electrode apparatus 400 includes a plurality of electrode portions (two are shown) 403, 405. In FIG. 3H, a front perspective view is shown. The front side is the side that will face away from the subject when worn. The cantilever electrode apparatus is thin, so that the electrode portions include a front side (visible in FIGS. 3H and 31) and a back side (visible in FIG. 3K). As shown in the side view of FIG. 3J, the device has a thin body that includes the electrode portions 403, 405 as well as an elongate body region 407 extending between the two electrode portions. The elongate body is also thin (having a much larger diameter and height than thickness). The thickness is shown in FIG. 3J.

In this example, two connectors 415, 417 (electrical and mechanical connectors, shown in this example as snaps) extend from the front of the cantilever electrode apparatus. The front of the first electrical portion 403 may also include an optional foam and/or adhesive material 421 through which the snaps extend proud of the first electrical portion. The first electrical portion is shaped and sized so that the snaps will connect to plugs (ports, holders, opening, female mating, etc.) on the electrical stimulator. As described above, the connectors may be separated by between about 0.6 and about 0.9 inches (e.g., between about 0.7 and about 0.8 inches, etc., shown in FIGS. 3H-3K as about 0.72 inches). The second electrode portion may also include a foam or backing portion 423. This foam/backing region may be optional. In some variations the separation between the connectors is not limited to 0.7 to 0.8, but may be larger (e.g., between 0.7 and 1.2 inches, 0.7 and 1.1 inches, 0.7 and 1.0 inches, 0.7 and 0.9 inches, etc.) or smaller (e.g., between 0.2 and 0.7, 0.3 and 0.7, 0.4 and 0.7, 0.5 and 0.7, 0.6 and 0.7 inches, etc.).

FIG. 3K shows a back view of this first example of a cantilever electrode apparatus. In this example, the first 403 and second 405 electrode portions are also shown and include active regions 433, 435. The active regions are bordered by adhesive 440. The first 403 electrode portion includes, on the back (patient-contacting) side, a first active region 433, which is bounded, e.g., around its entire circumference, or at least on, by an adhesive 440. The active region may include a conductive material (e.g., electrically conductive gel). Similarly, the back of the second electrode portion 405 includes the second active region 435 surrounded on two sides by an adhesive material 440 that extends to the edge of the electrode region. The adhesive may be any biocompatible adhesive that can releasably hold the material to the skin.

In general the elongate body region connecting the two electrode portions may be any appropriate length, but is generally longer than a few inches (e.g., longer than about 2 inches, longer than about 3 inches, longer than about 4 inches, longer than about 5 inches, longer than about 6 inches, longer than about 7 inches, longer than about 8 inches, longer than about 9 inches, etc.). The elongate body region may also be bent or curved, as illustrated in FIGS. 3H-3K. The bend or curve, in which the elongate body may even double back on itself, may allow the material to flex or bend to allow it to be adjustably positioned over and/or around the subject's head, as shown in FIGS. 3L and 3M, for example.

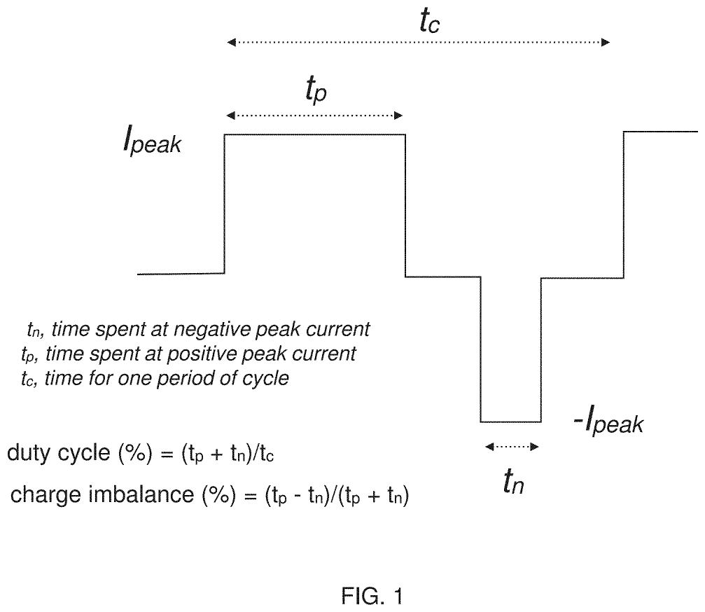

FIG. 3L illustrates a cantilever electrode apparatus (similar to those shown in FIGS. 1A and 4A) worn on a subject's head. As illustrated, the apparatus is positioned with the first electrode portion adhesively attached at the temple region and a second electrode portion attached to a region behind the head (e.g., neck region, not shown). A neurostimulator (not shown in FIG. 3L) may be attached to the cantilever electrode apparatus either before or after it is applied to the subject. As shown in FIG. 3M, the neurostimulator may be attached to the front side of the cantilever electrode apparatus by snapping onto the proud connectors, while the elongate body region 407 is bent to extend behind the subject's head and down to a portion on the midline of the back of the patient's neck. Both the first electrode portion and the second electrode portion may be adhesively held with the electrically active regions against the skin, allowing the neurostimulator to apply energy, and in particular the waveforms as described in U.S. application Ser. No. 14/320,443, titled “TRANSDERMAL ELECTRICAL STIMULATION METHODS FOR MODIFYING OR INDUCING COGNITIVE STATE,” filed Jun. 30, 2014, and herein incorporated by reference in its entirety.

In use, a user may interact with a controller (e.g., a smartphone controlled by application software/firmware) that pairs with the neurostimulator (e.g., by Bluetooth).

An ensemble waveform may generally be between about 3-90 min (e.g., between about 3-60 min, between about 5-60 min, between about 5-40 min, etc., between about 3-25 minutes, etc.) long, or longer (e.g., greater than 3 min, greater than 5 min, greater than 10 min, greater than 12 min, etc.). In general, an ensemble waveform may be broken up into segments with specific pulsing parameters, e.g., current amplitude, frequency, duty cycle, charge imbalance, shorting/capacitive discharge, etc., and these parameters may change at pre-specified times for subsequent component waveforms. Once the user selects an ensemble waveform, and the non-invasive neuromodulation waveform is added to the patient's device, the patient can start the neurostimulation and the user can control or change the perceived intensity (e.g., by dialing the perceived intensity up or down), pause, or stop the session using the phone (app). In general, the perceived intensity can be scaled by the user between 0-100% of a target perceived intensity (e.g., a target current, frequency, duty cycle, charge imbalance, and/or shorting/capacitive discharge), using a control such as one or more buttons, sliders, dials, toggles, etc., that may be present on the controller (e.g., smartphone) in communication with the neurostimulator. In addition, the controller may be configured to allow the user to press an icon to help in applying the electrode apparatus and/or neurostimulator. For example, activating this control may cause the smartphone to activate a front-facing camera on the phone to help the user to attach the apparatus to the head. During or after a session, a user can access help screens, a profile page, feedback about a session, and analysis & history of previous use. In general, the system may also be configured to pass data to and from the controller and/or the neurostimulator and to/from a remote server via the Internet. These data may include user information, subject/patient information, compliance data, dosage information (e.g., waveform data), information about the function or state of the hardware device or electrode assembly, etc.

The neurostimulator may apply a non-invasive neuromodulation waveform for about 3-30 min (or longer) that is made up of different “blocks” having repeated waveform characteristics; the waveform ensemble may include transition regions between the different blocks. In general, at least some of the waveform blocks (and in some variations most or all of them) generally have a current amplitude of >3 mA (e.g., between 5 mA and 40 mA, between 5 mA and 30 mA, between 5 mA and 22 mA, etc.), and a frequency of >100 Hz (e.g., between 250 Hz and 15 kHz, between 750 Hz and 25 kHz, between 750 Hz and 20 kHz, between 750 Hz and 15 kHz, etc.), the current is typically biphasic and is charge imbalanced, and has a duty cycle of between 1-90% (e.g., between 10-90%, between 30-80%, between 30-60%, etc.). One or more of these characteristics may be changed during stimulation over timescales of every few seconds to minutes. FIG. 1 shows an exemplary cycle of a waveform for non-invasive neuromodulation comprising a positive-going pulse of duration tₚ, a negative-going pulse of duration tₙ, and a total pulse duration of t_{c}. As shown in FIG. 1 the peak of the positive- and negative-going pulses may be equal (absolute value). The duty cycle percentage may be defined as (tₚ+tₙ)/t_{c} and the charge imbalance percentage may be defined as (tₚ−tₙ)/(tₚ+tₙ).

In general, the non-invasive neuromodulation control module may be specifically adapted to deliver a biphasic electrical stimulation signal of 10 seconds or longer between the first and second electrodes, where the signal has a frequency of 100 Hz or greater (e.g., 200 Hz or greater, 400 Hz or greater, 450 Hz or greater, 500 Hz or greater, 600 Hz or greater, 700 Hz or greater, etc.; optimally 750 Hz or greater, including 1 kHz or greater, 2 kHz or greater, 3 kHz or greater, 4 kHz or greater, 5 kHz or greater, 7.5 kHz or greater, 10 kHz or greater, 20 kHz or greater, etc.) and an intensity of 2 mA or greater (e.g., 3 mA or greater, 4 mA or greater, 5 mA or greater, 6 mA or greater, 7 mA or greater, 8 mA or greater, 9 mA or greater, 10 mA or greater, etc.). The control module may also be configured to reduce pain when applying the stimulation by controlling the duty cycle (e.g., the percent of time that the current applied is non-zero, and/or greater than zero), e.g. so that the duty cycle of the applied energy is greater than 10 percent (e.g., greater than 15 percent, greater than 20 percent, greater than 30 percent) and less than 90 percent (e.g., less than 75 percent, greater less than 70 percent, less than 60 percent). In addition, the control module may be configured so that the applied current is biphasic and/or is not charge balanced (e.g., has a DC offset, also referred to as DC bias, so that the mean amplitude of the applied waveform is non-zero). Alternatively or in addition, the control module (non-invasive neuromodulation control module) may be configured to deliver waveforms biphasically asymmetric (i.e., not having the same pulse in the positive and negative direction) and/or to discharge capacitance built up on the electrodes (and in the body), e.g., by occasionally or periodically “shorting” the electrodes, and/or by applying an opposite current(s). In general, a control module may be configured to generate stimulation that includes these parameters, and may be configured to prevent stimulation outside of these parameters, in order to avoid inducing pain.

Described herein is a method of treating psoriasis, including facilitating a suppression of sympathetic tone for a predetermined period. Such methods may generally include: placing, on a patient suffering from psoriasis, a first and second electrode of a wearable non-invasive neuromodulation applicator on the subject's skin; activating the wearable non-invasive neuromodulation applicator to deliver a non-invasive neuromodulation stimulation having a duty cycle of greater than 10 percent (e.g., greater than 15%, etc.), a frequency of 250 Hz or greater, and an intensity of 3 mA or greater. The first electrode and second electrode may be placed together (as part of a single pad, patch or applicator) or separately. The first electrode may be placed in a first region (e.g., on a neck); the second electrode of the non-invasive neuromodulation applicator may be placed on a second location (e.g., on the back of the subject's neck above the vertebra prominens, on the skin over the C7-T2 region of the spine, etc.). The biphasic non-invasive neuromodulation electrical stimulation may be asymmetric with respect to positive and negative going phases; and facilitating the treatment of psoriasis by applying the biphasic non-invasive neuromodulation electrical stimulation between the first and second electrodes for 10 seconds or longer.

Also described herein are methods of treating psoriasis in a subject in need thereof, which may include: placing, on the skin of a subject suffering from psoriasis, the first and second electrodes of a wearable non-invasive neuromodulation applicator on the subject's skin (e.g., on a temple region on a first side of the subject's body, and/or on the back of the subject's neck, etc.); activating the wearable non-invasive neuromodulation applicator to deliver a non-invasive neuromodulation electrical stimulation having a duty cycle of greater than 10 percent, a frequency of 250 Hz or greater, and an intensity of 3 mA or greater. The stimulation may be biphasic non-invasive neuromodulation electrical stimulation that is asymmetric with respect to positive and negative going phases. The method may generally include treating psoriasis by applying the biphasic non-invasive neuromodulation electrical stimulation between the first and second electrodes for 10 seconds or longer.

In any of these methods the subject may be concurrently taking a drug (topical and/or systemic) for treating their psoriasis. These methods may therefore accelerate, enhance or improve the drug effect(s), and/or allow a smaller dosage to be taken.

As mentioned above, any of the portable non-invasive neuromodulation applicators descried herein for treating psoriasis in a subject may include: a body; a first electrode; a second electrode; and a non-invasive neuromodulation control module at least partially within the body and comprising a processor, a timer and a waveform generator, wherein the non-invasive neuromodulation control module is adapted to deliver a biphasic electrical stimulation signal of 10 seconds or longer between the first and second electrodes having a duty cycle of greater than 10 percent, a frequency of 250 Hz or greater, and an intensity of 3 mA or greater, wherein the biphasic non-invasive neuromodulation electrical stimulation is asymmetric with respect to positive and negative going phases.

For example, a wearable non-invasive neuromodulation applicator may include: a body; a first electrode; a second electrode; a non-invasive neuromodulation control module at least partially within the body and comprising a processor, a timer and a waveform generator, wherein the non-invasive neuromodulation control module is adapted to deliver a biphasic electrical stimulation signal of 10 seconds or longer between the first and second electrodes having a duty cycle of greater than 10 percent, a frequency of 250 Hz or greater, and an intensity of 3 mA or greater, wherein the biphasic non-invasive neuromodulation electrical stimulation is asymmetric with respect to positive and negative going phases; and a wireless receiver connected to the non-invasive neuromodulation control module; wherein the wearable non-invasive neuromodulation applicator weighs less than 50 grams.

Any of these apparatuses may be specifically adapted for use to treat psoriasis. For example, in some variations, the apparatus includes one or more sensor that determine the sympathetic/parasympathetic state (e.g., sympathetic tone) of the subject wearing the apparatus. Sensors may include one or more accelerometers, heart rate sensors, electroencephalogram (EEG) sensors, electromyogram (EMG, including electrooculogram EOG), pulse oxygenation sensor(s), etc. As used herein, a sensor may also include hardware and/or software for interpreting and/or modifying the resulting signals, including but not limited to filtering physiological signals, amplifying physiological signals, etc. These sensors may be integrated into the apparatus of separate from the apparatus.

The methods and apparatuses (devices, systems) described herein may use a non-invasive neuromodulation waveform having one or more characteristics from the list including: a duty cycle between 30% and 60%; a frequency greater than 5 kHz or greater than 10 kHz; an amplitude modulation, including amplitude modulation with a frequency less than 250 Hz; and a burst mode wherein stimulation pauses intermittently (i.e., on for 100 ms, off for 900 ms; on for 500 ms, off for 500 ms; and other more complex pulsing patterns, including chirping and patterns repeating at 250 Hz or lower frequency).

Some versions of the methods and systems described herein include monitoring of the subject; this monitoring may be used as feedback into the apparatus to regulate the non-invasively applied neuromodulation waveform(s), and/or duration of application of the non-invasive neuromodulation. Monitoring may comprise using a sensor (which may be included as part of the apparatus or used along with the apparatus) to measure a subject's brain rhythms (i.e., EEG, including in particular alpha waves), autonomic function (including sensors to measure one or more of: galvanic skin resistance, heart rate, heart rate variability, or breathing rate, pulse oxygenation), and/or movements. Variations of the systems and methods described herein may further comprise an automatic modification of a non-invasive neuromodulation electrical stimulation waveform based on the collected (sensed) data. Thus, any of the apparatuses described herein may be configured to feed the sensor information back to control (e.g., turn on/off) and/or modify the non-invasive neuromodulation stimulation applied.

In some variations of the systems and methods described herein, a non-invasive neuromodulation waveform may be started, stopped, or modified based on sensor data (e.g., and/or determined sympathetic tone) relative to a threshold value. In other variations of the systems and methods described herein, a non-invasive neuromodulation waveform may be started, stopped, or modified based on a measurement of the subject's physiology or cognitive state including but not limited to: activity, stress, immune system function, diet, and mood.

The systems and methods described herein may further comprise a notification that reminds the subject to wear a neurostimulator for a treatment period. For example, the notification to the subject may be based on input from a location sensor in the neurostimulator or a device wirelessly connected to the neurostimulator and a clock. In other embodiments, the system or method may further comprise a calming sensory stimulus (i.e., an auditory stimulus, including binaural beat, and olfactory stimuli).

The non-invasive neuromodulation waveforms that may be applied (e.g., to the subject's neck or head and neck) to treat psoriasis as described herein include a range of parameters that may be adjusted for both efficacy and comfort. The data described herein suggest that in some variations it may be beneficial to provide relatively low frequency (e.g., 250 Hz to 750 Hz, 250 to 1 kHz, 250 to 3 kHz, 250 to 5 kHz, etc.) stimulation at relatively high current (e.g., >3 mA, greater than 4 mA, greater than 5 mA, etc.); however these two parameters alone, low frequency and high current, typically result in painful and/or unpleasant sensations on the head and/or neck when applied there. In order to achieve a combination of low (250-750 Hz) frequency and high current (>3 mA, 3-40 mA, >5 mA, etc.) it may be beneficial to include one or more of the modulation schemes described herein, including DC offset (biphasic, asymmetric stimulation in which the positive and negative going pulses are different durations and/or amplitudes), percent duty-cycles (e.g., between 10-80%, etc.) and the use of an AC (carrier) frequency (<250 Hz). In some variations, the use of just one or two of these modulation schemes may be sufficient (e.g., using just a DC offset and a percent duty cycle between 10-80%, or just a DC offset and an AC carrier frequency <250 Hz, or just a percent duty cycle between 10-80% and an AC carrier frequency of <250 Hz), while in some variations, all three may or must be used.

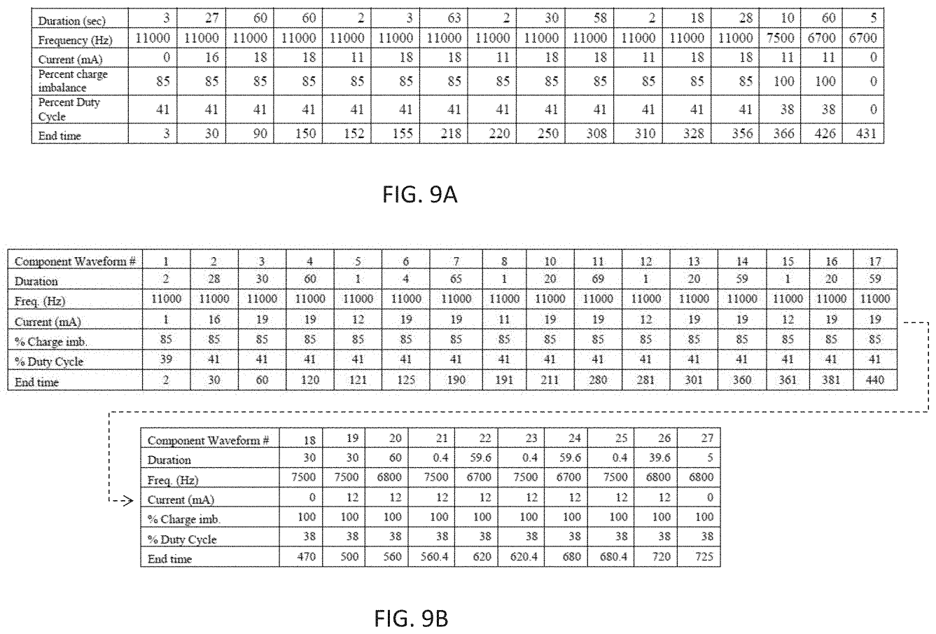

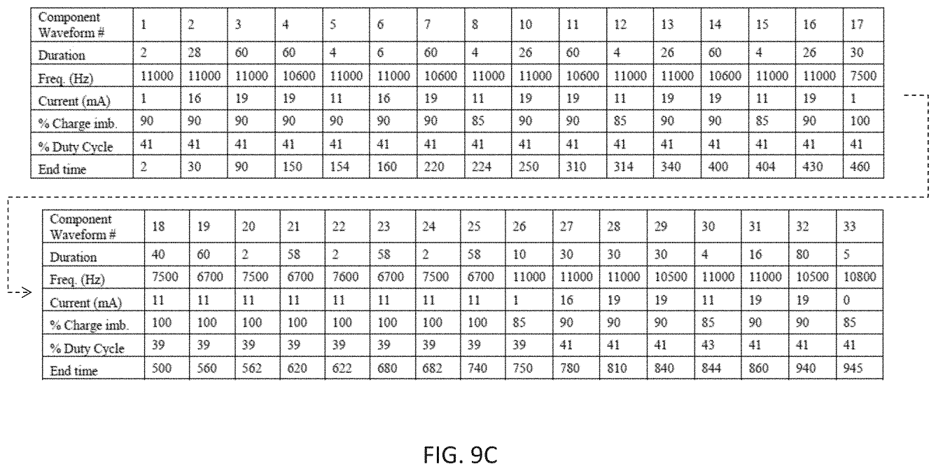

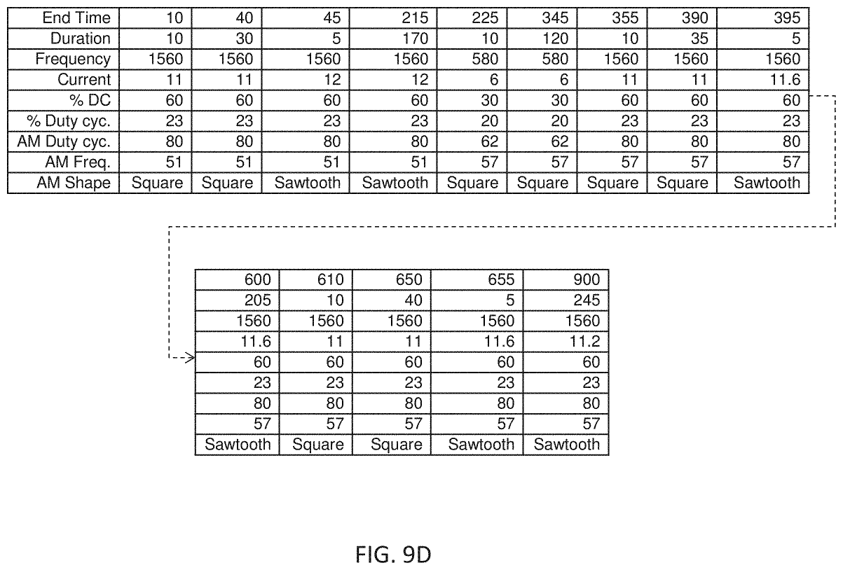

In general, any appropriate waveform may be used. For example, one waveform ensemble that may be used is referred to as ‘high F’ (or alternatively as ‘Program B’ or relaxCES) and is a pulsed waveform with variable frequency, generally between 3 kHz and 11 kHz. FIGS. 9A-9C describe three example of complete ensemble waveforms that may be similar to the “high F” non-invasive neuromodulation waveforms used.

The tables shown in FIGS. 9A-9C lists the waveform parameters for each of the component waveforms. In this example the ensemble waveform is configured with short circuiting on (meaning that a capacitive discharge pulse occurs in the opposite direction after each of the biphasic pulses). In one example, the system transfers chunks (e.g., 400 ms segments) securely between the user device and the worn neurostimulator every about 400 ms (or on multiples of about 400 ms), including the neuro stimulation start frequency, end frequency, starting amplitude, end amplitude, start duty cycle, end duty cycle, start percent charge imbalance, end charge imbalance, etc. The timing of wireless communication chunks at about 400 ms should not be construed as limiting the timing of communication between a controller unit and the neurostimulator. FIG. 9B illustrates a second example of a calm ensemble waveform having a slightly longer running time, running over 12 minutes. Similarly, 9C illustrates a third example of a calm ensemble waveform having a yet longer running time (over 16 minutes).

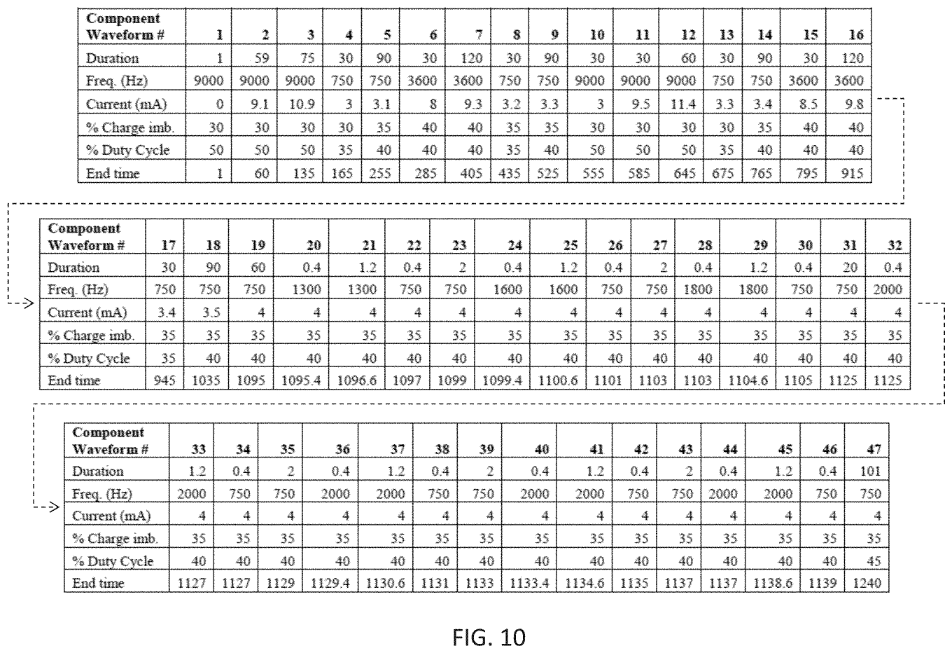

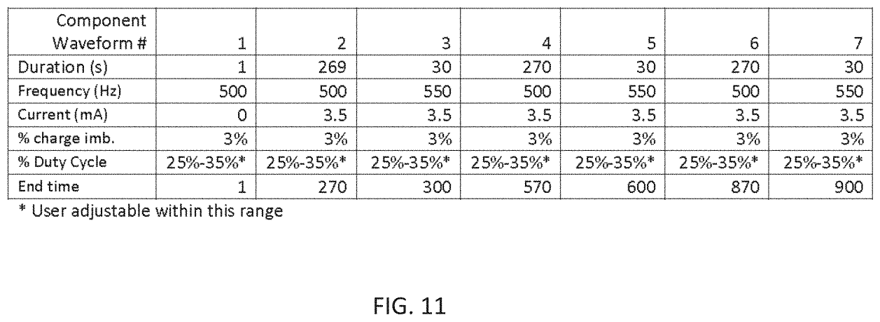

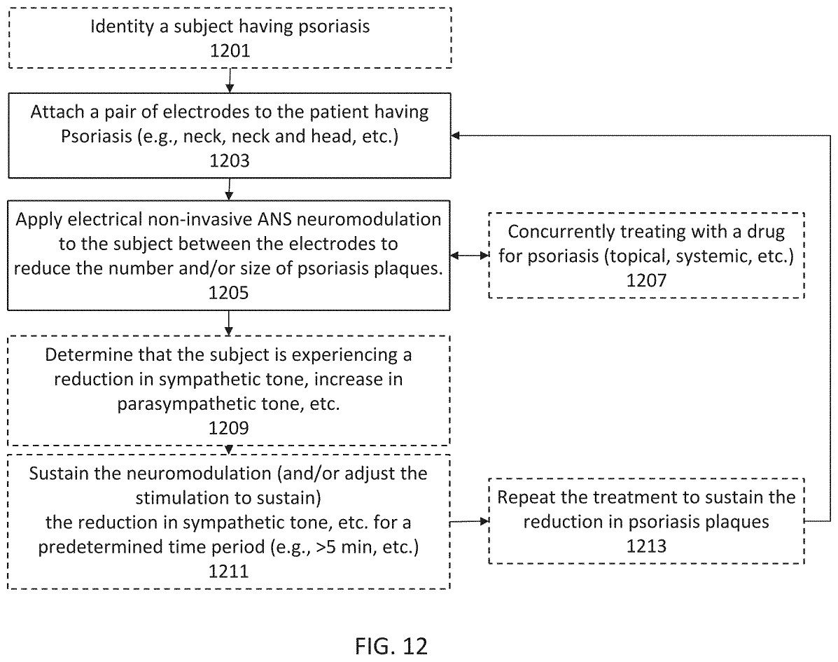

A second waveform is referred to as ‘low F’ (or alternatively as ‘Program A’). This second waveform has a lower pulsing frequency, variable but generally 750 Hz. FIG. 10 illustrates an example of a non-invasive neuromodulation ensemble waveform such as the low F variations described herein.

The use of non-invasive neuromodulation to modulate neural activity is supported by a long history of safety obtained over four decades. There are numerous methods and devices intended for modulating peripheral nerve structures using transcutaneous delivery of voltage/current waveforms from electrodes applied to various locations on the body. These devices such as transcutaneous electrical nerve stimulation (TENS), powered muscle stimulation (PMS), electrical muscle stimulation (EMS) and others have amassed such a high degree of physical safety that they have been moved to an over-the-counter product rather than a medical device requiring a prescription depending on the intended use and design characteristics. For example, legally marketed electrical nerve stimulation devices are already commercially available and have output levels far greater than the ones we implemented here. These devices intended for over-the-counter cosmetic applications of TENS target similar anatomical regions and nerve targets such as the trigeminal nerve. One example is an over-the-counter cosmetic TENS device (Bio-medical Research Face), which is designed to target the trigeminal nerve and provide neuromuscular electrical stimulation (NMES) to encourage facial rejuvenation for aesthetic purposes. A recent study examined the safety and efficacy of this device at a peak current intensity (35 mA) that was nearly twice the one used in our study when used five days per week for 20 minutes each day for 12 weeks. There were no significant adverse events in this study and the only reported side effects were minor skin redness following stimulation, which disappeared with 10-20 minutes following use. Another device, which modulates supraorbital branches of the trigeminal nerve to treat headache has also demonstrated a high safety threshold when used daily for multiple weeks. Other reports using trigeminal nerve stimulation for the treatment of epilepsy, depression, and other disorders have likewise shown a high degree of safety. Although there is a high degree of confidence in the safety of trigeminal nerve modulation, caution is always warranted when delivering electrical currents to the human body and we advise investigators to learn and implement safe practices using qualified devices.

### EXAMPLES

Subject's having psoriasis (e.g., having a Psoriasis Area and Severity Index, or PASI score indicating mild to severe psoriasis) were treated as described herein. Improvement were generally seen in patient's treating for 3 or more times per week (e.g., treating daily or every-other day for at least 10 minutes per treatment), with greater improvement seen with daily and >10 min/day usage. It typically took between 7-10 days for improvements to being to manifest, where the improvements included a reduction in itchiness, area affected and overall skin quality. Thus, methods of treating a patient as described herein may include treating at least once every 48-60 hours for at least 10 minutes in any of the waveforms described herein (see, e.g., FIGS. 7-9), where the treatment continued for at least one week (e.g., at least two weeks, at least three weeks, at least four weeks, etc.).

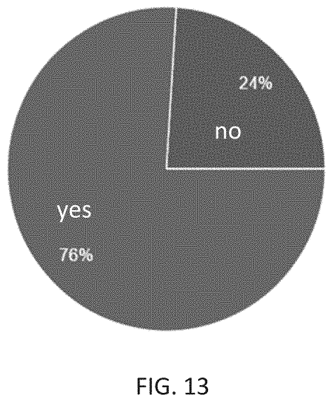

The methods and apparatuses described herein, including the use of the neurostimulators and waveforms, for treatment of inflammatory disorders such as psoriasis may be due to the reduction in stress. The neurostimulation programs described herein have been found to significantly decrease salivary amylase acutely and after 7 days of use compared to placebo. These neurostimulation programs have also been found to acutely lower tension and anxiety by ˜20% using a Profile of Moods Scale. After 7 days, subjects reported a ˜41% reduction in stress and a ˜30% reduction in anxiety compared to placebo using Depression Anxiety Stress Scale. A multicenter review showed Xanax led to a 44% decrease in stress using a similar scale. Using a within groups comparison of subjects tested with the placebo and real program, only 8% of subjects thought the placebo had a stronger effect (p=1.7×10⁻²⁰). Further, the neurostimulation programs described herein significantly affect Heart Rate Variability compared to placebo treatment, and also suppress Galvanic Skin Conductance by 53% compared to placebo treatment in a fear conditioning paradigm. These neurostimulation programs also show an effect size on stress of 0.67. Benzodiazepenes have varying strengths but a review of the literature shows an overall effect size of 0.38 with commonly used doses. These neurostimulation programs also lead to a significant improvement in sleep quality as measured by the Pittsburgh Sleep Quality Index and clinical sleep actigraph, including a 37% reduction in middle of the night awakenings compared to placebo treatment. FIG. 13 is a pie chart showing the results of a survey of 89 “high-need” users of the device (e.g., users having reported often feeling stress/anxiety, and/or users sleeping less than 5 hours/night, self-reported). Approximately three quarters of these users reported sleeping better and/or having lower stress/anxiety following (on average) 12 sessions of the use of the neurostimulator and waveforms described herein.

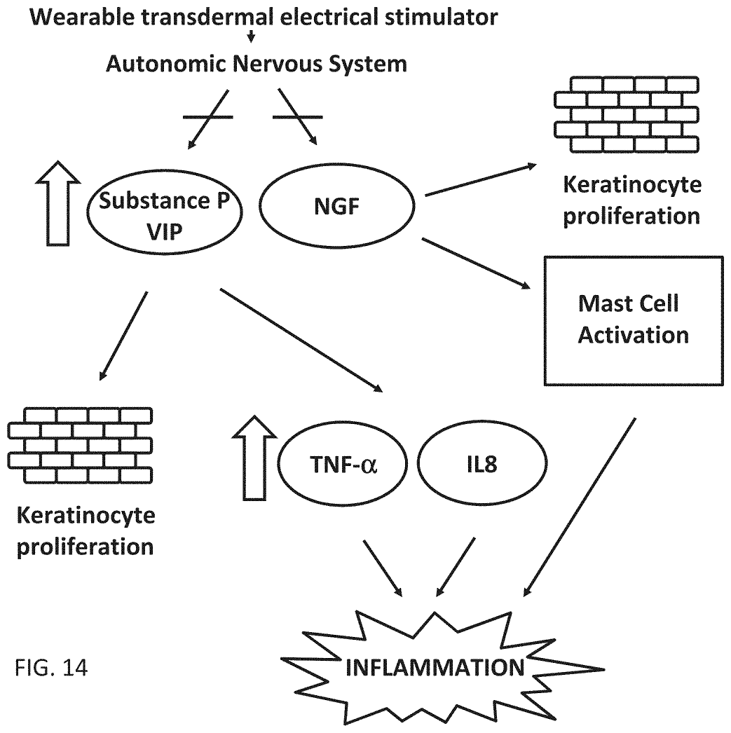

FIG. 14 is a schematic illustration of one possible theory of operation for the reduction in inflammation (and therefore treatment of, in this example, psoriasis) using the methods and apparatuses descried herein. This possible mechanism of action is not intended to be limiting, and the methods described herein may be effective even if this mechanism of action proves inaccurate. In FIG. 14, the neurostimulator apparatus may apply one or more of the waveforms described herein to a subject. This non-invasive neuromodulation may significantly modulate activity and suppress the stress response. An increased autonomic response may lead to increased levels of Substance P, VIP and NGF at the skin. Substance P and VIP are known to have a stimulatory effect on keratinocyte proliferation while upregulating TNF-alpha, IL1 and IL8. NGF is known to promote keratinocyte proliferation as well as mast cell degranulation. An anti-NGF topical therapy has been shown in other contexts to reduce PASI and control symptoms of itch. Further, traumatic nerve injury leads to a remission of psoriasis, a phenomenon thought to be mediated by a decline in neuropeptides. Repeated injections of local anesthetic, which prevents the release of neuropeptides, can lead to plaque clearance. Thus, the methods described herein may similarly inhibit Substance P/VIP and/or NGF increases leading to TNF alpha, IL8 and mast cell activation, and subsequently, inflammation, thereby improving inflammatory disorders such as psoriasis.

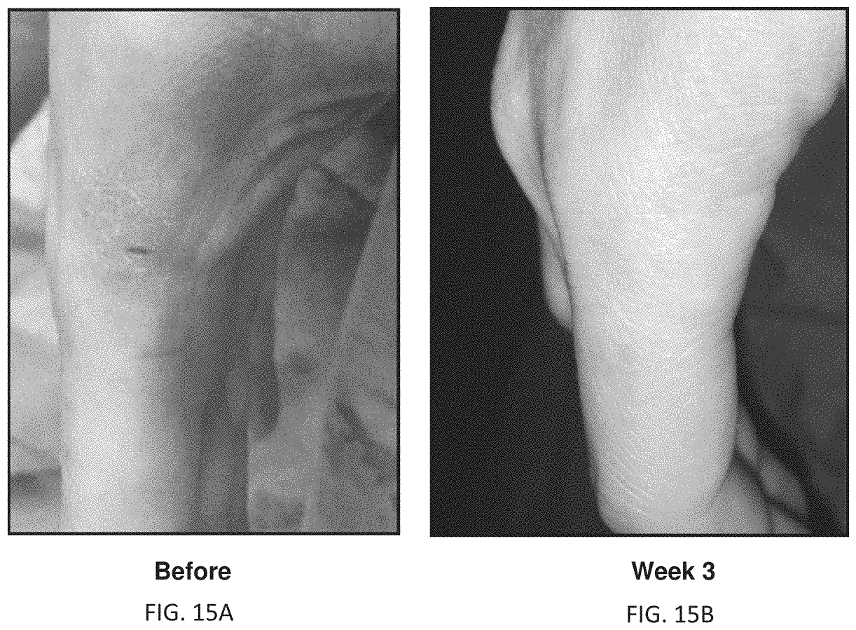

FIGS. 15A-15B, 16A-16B and 17A-17B illustrate images typically of the improvements in psoriasis lesions resulting from the methods and apparatuses described herein. In FIG. 15A-15B, a female user experiencing mild psoriasis with stress-related flares, not taking any other medications, showed a substantial improvement in overall psoriasis. FIG. 15A shows a lesion (on the subject's hand), before treatment, and in FIG. 15B, following 3 weeks with multiple treatment sessions (30 sessions). The user reported a reduction in the size (by half) and reduction in itching.

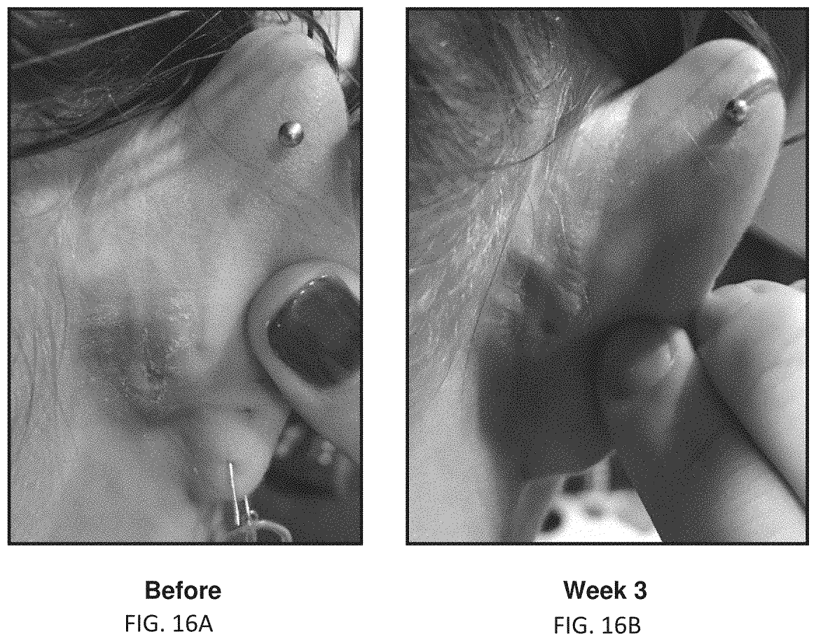

FIGS. 16A-16B show before and after images for another female subject experiencing moderate psoriasis. In FIG. 16A a lesion located behind the ear is shown before any treatment; in FIG. 16B the same region of skin is shown following 3 weeks with 12 sessions. Overall, the patient's (user's) lesions have improved significantly. The patient experienced a relapse in lesions when not regularly using the apparatus and methods described herein.

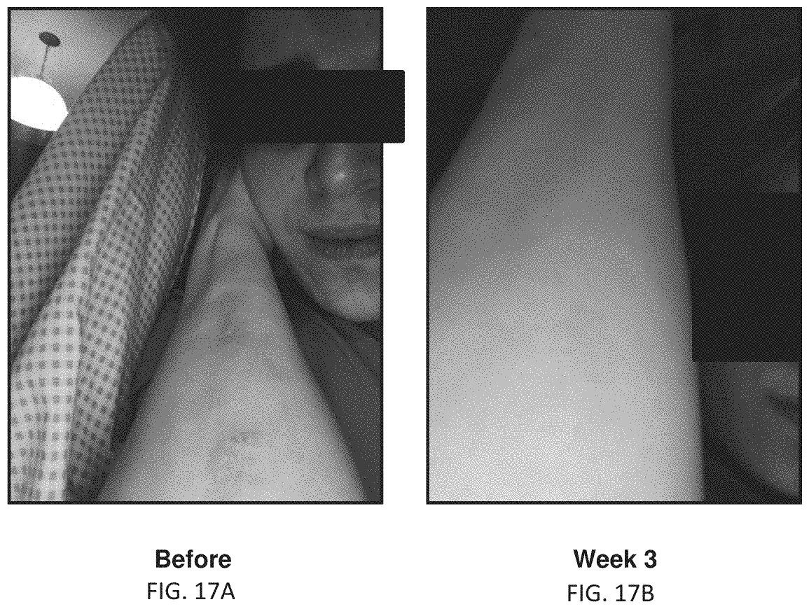

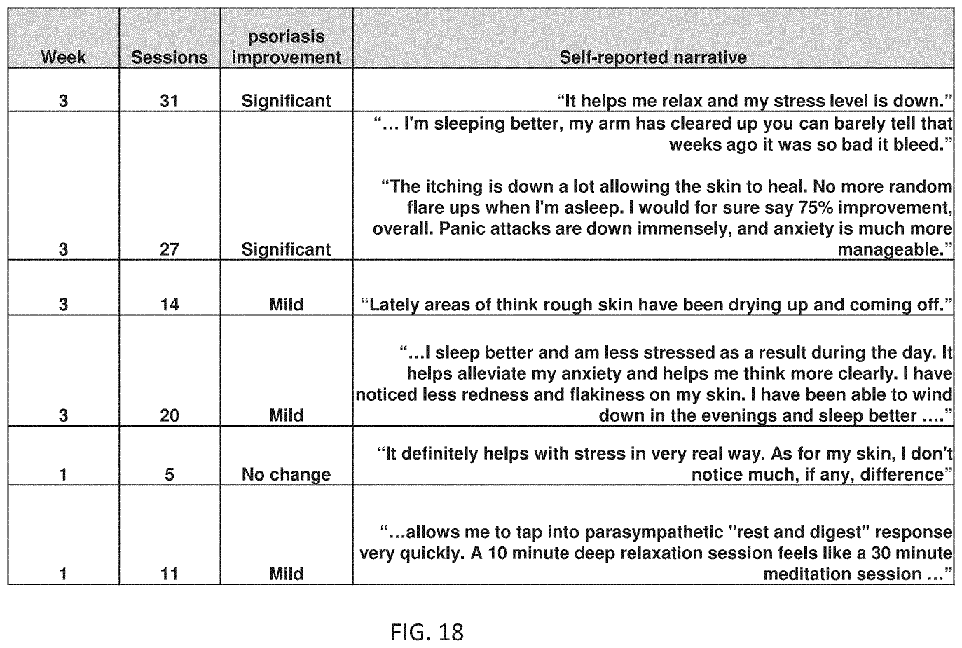

FIGS. 17A-17B illustrate the elimination of a patient's psoriasis lesions following 29 sessions over three weeks. This patient normally experiences moderate psoriasis, described as “extremely painful and itchy” when untreated. Following treatment with the methods and apparatuses described herein, a significant reduction in the number and extent of lesions was reported. FIG. 17A shows multiple lesions on the subject's right arm; in FIG. 17B the lesions have been eliminated following the 3 week treatment period.

FIG. 17 illustrates preliminary results of a pilot study looking a treatment of psoriasis as described above. In FIG. 17 four patients/users have undergone at least 3 weeks of treatment, and reported mild to significant improvement in their psoriasis during the treatment period (between 14-32 sessions). Self-reported data shows a substantial improvement in quality of life. Data is also shown for two newer subjects at the first week of treatment (5-11 sessions), showing mild or no change. These subjects will be monitored for at less three total consecutive weeks to track any changes. Overall, subjects suffering from psoriasis typically experience mild to profound improvements, including a reduction in the number of lesions, the extent of the lesions and the irritation of the lesions.

In general, any of the apparatuses described herein may include a user interface that is adapted for the treatment of psoriasis. For example, in general, any of these apparatuses may include a user interface that presents (on the wearable stimulator itself or a smartphone, tablet or other processor in communication with the wearable stimulator) the patient with a control to start and stop the application of the non-invasive neuromodulation. The control may include a timer and/or calendar for scheduling. The interface may also include one or more inputs for allowing the subject to input information, such as self-reported information about the severity and extent of their psoriasis (e.g., lesions, redness, itchiness, etc.). The interface may present a body map, showing a schematic of listing of the body regions (head, torso, arms, legs, chest, back, buttocks, etc.) for the various parts of the users body, and the user may select one or more body regions and input information about the psoriasis specific to that region. The input may allow for images (e.g., using the smartphone camera, particularly but not exclusively when the user interface is executing on the user's smartphone and communicating with the non-invasive neuromodulation stimulator) showing lesions. The apparatus may store this information for tracking progress of the therapy.

Thus, in general the apparatus may include a user interface (e.g., application software/firmware/hardware) that allows control of the application of non-invasive neuromodulation waveforms, including start/stop, adjustment to the intensity, selection between various psoriasis-specific waveforms, duration control, etc. The apparatus' user interface may also include assessment inputs, for tracking the extent and/or degree of the user's psoriasis, as mentioned above. In some variations the user interface may also present a ranking or score of the user's psoriasis based on the input (e.g., using a scoring system such as the PASI). This tracking or assessment information may also be stored and/or transmitted to a physician or health care provider, or third-party (e.g., at a remote processor).

The user interface may also allow tracking of the treatment and dosage. For example, the user interface may provide reminders for the next prescribed or scheduled dose(s), and may preselect the dose time and waveform(s). The user interface may prompt the user to apply the therapy, and/or to apply or remove the electrode pad/patch and non-invasive neuromodulation stimulator. For example, the apparatus may include control logic that prompts for dosing of 3× per week (e.g., every 24 hours, every 24 hours, every 60 hours, etc.) for at least a week, with treatment sessions of greater than 10 minutes.

The apparatus, including the application (e.g., user interface) portion of the apparatus may be software that is executable on a processor that is part of the wearable apparatus of in wireless communication with the wearable, for example on a user's smartphone, laptop, tablet, smartwatch, or other wearable electronics or the like. The application portion of the apparatus may also receive any of the inputs described above (e.g., for tracking sympathetic or parasympathetic activity or tone).

Although the methods and apparatuses described herein are described specifically with respect to psoriasis, any of these method and apparatuses may also be used to treat other skin disorders and particularly skin disorders are inflammatory or auto-immune in nature. For example, these methods and apparatuses may also or alternatively be used to treat one or more of: acne, dermatitis (eczema), scleroderma, dermatomyositis, epidermolysis bullosa, and bullous pemphigoid.

In general, any of the apparatuses described herein (e.g., within the processor of the neurostimulator) may include firmware and communication protocols for receiving and responding to the command messages. Any of the processors (neurostimulators) described herein may also be configured to transmit error codes back to the controller. For example, the processor may, during communication (e.g., via a communication circuit) check whether received waveform parameters comply with limitations of hardware and safety standards. Examples of error codes that may be safety conditions (e.g., current requested too high, electrode contact lost or poor connection, DC limit reached, communication lost), error codes related to the received command messages/communication (e.g., too many wave segments, fewer segments received than expected, received segments too short, received segments too long, etc.).

Any of the apparatuses for neurostimulation described herein may be configured to receive a plurality of neurostimulation command messages, including in particular the new waveform message and subsequent segment messages, which may include parameters from a controller such as a computing device (e.g., smartphone, etc.) and apply them as stimulation. The neurostimulator may also adjust them and/or send one or more response error messages back to the controller if the parameters contained in the messages do not comply with hardware limitations and/or safety limits which may be included in the neurostimulator.

### Example 2

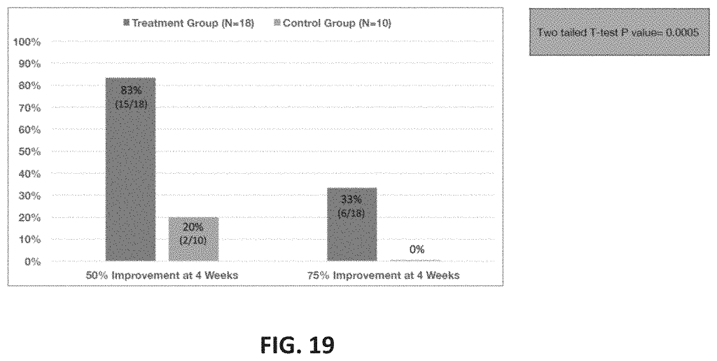

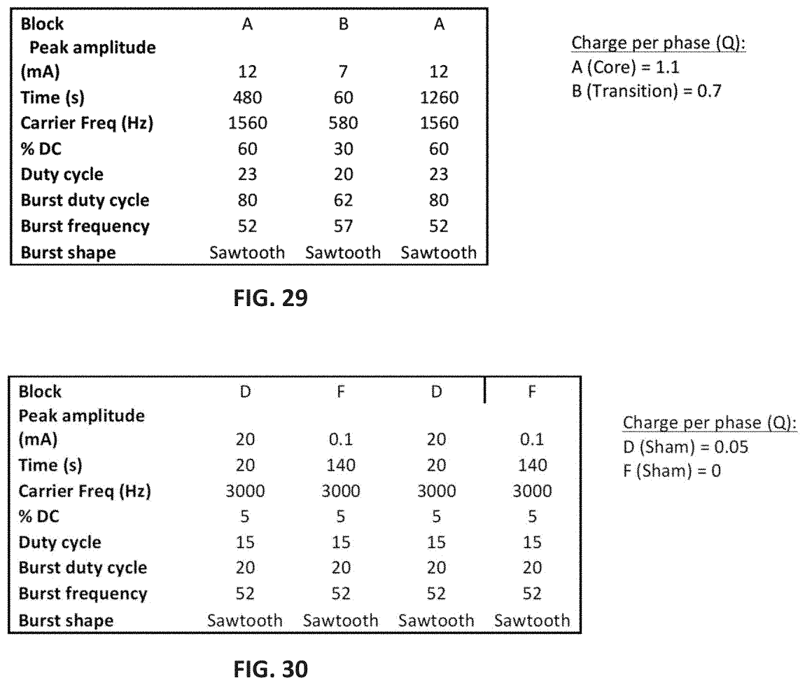

FIGS. 19-23 illustrate the results of a pilot study using N=18 treatment patients and N=10 control patients that had severe, moderate, or mild plaque psoriasis. Subjects used neurostimulation as described herein (e.g., using a waveform regimen similar to that shown in FIG. 29, for treatment, or 30, for sham control). Stimulation was applied 1× daily for at least 10 minutes, and weekly surveys and photographs were used to record data. Survey data indicated reported improvement in appearance (redness/scaling), itchiness, and anxiety levels. Overall, significant improvement was measured as greater than or equal to 50% improvement in appearance after 4 weeks. 90% of the subjects used topical treatments (in both groups) to treat.

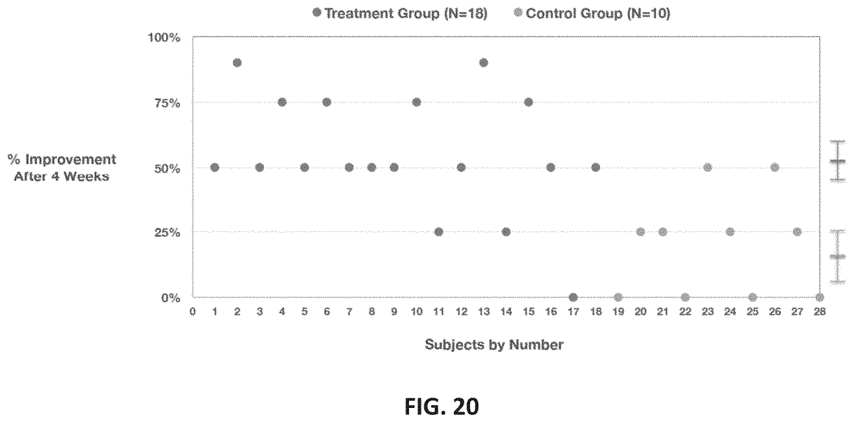

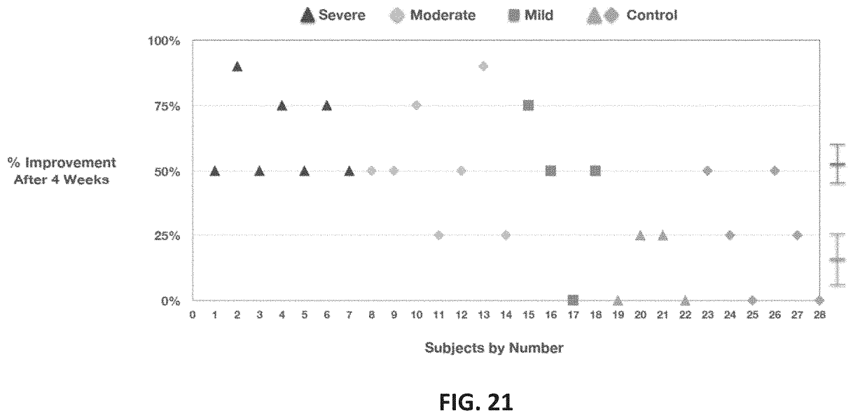

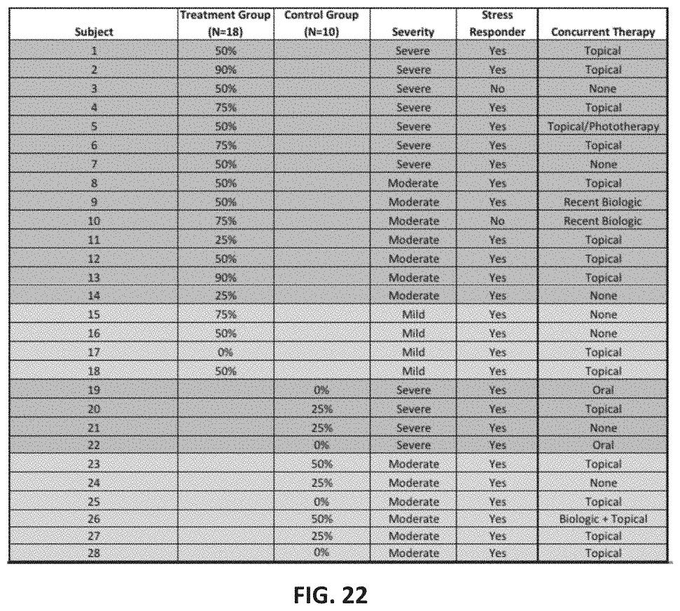

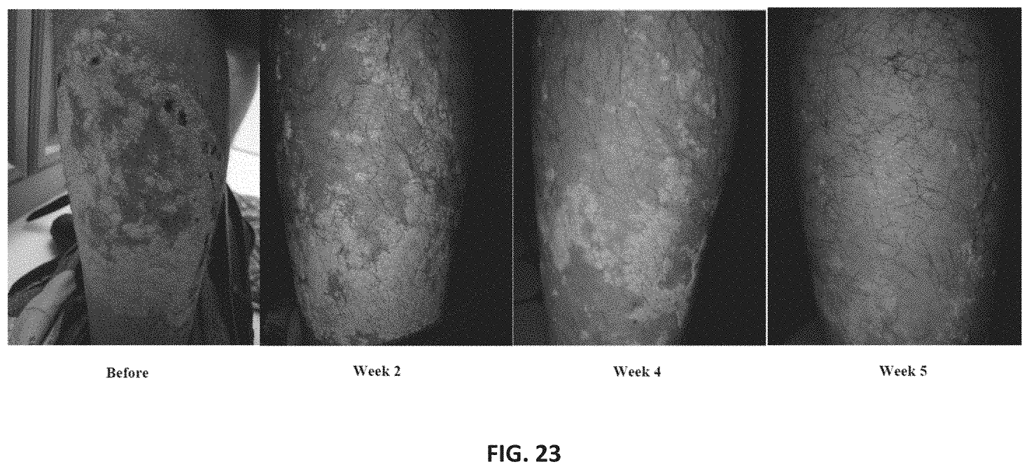

As shown in FIG. 19, 15 of the 18 patients in the treatment group had a 50% or greater improvement after 4 weeks. 6 of the 18 had a 75% or more improvement after 4 weeks. This was highly significant, compared to sham control. FIGS. 20-21 show where individual patients in both groups fall; patients 1-18 were treatment, patients 19-28 were control. This data is shown in tabular form in FIG. 22. FIG. 23 illustrates treatment effect for one patient, initially having moderate plaque psoriasis, over 5 weeks, showing a dramatic improvement by week 5. Similar results were seen with scalp (including sever) psoriasis.

### Example 3

In another example, a neck-worn apparatus, such as shown in FIGS. 31A-34B may be used to treat an autoimmune disorder, such as psoriasis. For example, the apparatuses and methods described herein may be intended for patients with moderate to severe plaque psoriasis who are candidates for phototherapy or systemic therapy. In general these neurostimulation apparatuses, including the system shown in FIGS. 31A-34B may be a portable (wearable), battery-powered, electrical neuromodulation device configured to provide a systemic treatment for moderate-to-severe psoriasis. The device may deliver a pre-programmed low-intensity, non-invasive neuromodulation electrical stimulus to the base of the neck and may be used with an accompanying software (e.g., application user interface or “app”) on a portable device, such as a smartphone. In some variations the system may include a soft neckband having attachment points for one or more disposable gel electrode assemblies (e.g., “gel pads”) to be positioned at the base of the neck (e.g., the C3 to C7 region) and the neurostimulator at the front. This configuration may allow the system to be worn and applied comfortably at the treatment site.

The system may be pre-programmed to deliver a specific treatment regimen (including a specific waveform) for a specific duration. The waveform settings may not be accessible to the subject (e.g., patients). In this example, the apparatus has only has one button, a power button. A subject may initiate a low-intensity, electrical stimulus for a treatment period, e.g., of about fifteen (15) minutes (e.g., about 5 minutes, about 7.5 minutes, about 10 minutes, about 12 minutes, about 15 minutes, about 17.5 minutes, about 20 minutes, etc.) using the user interface (app), which may be operated, for example, on a smartphone. The app may also signal the patient visually at the beginning and end of electrical stimulus. The wearable device may wirelessly communicate (e.g., via Bluetooth, Bluetooth Low Energy wireless protocol, etc.) with the app.

In some variations, the system may include: a neuromodulation device (e.g., neurostimulator, neuromodulator, etc.), a control program (e.g., including a user interface), which may be configured as an app, one or more gel pads (e.g., electrodes), and a neckband. Optionally, the system may include a data cord (e.g., USB cable) and/or a power adapter.

FIGS. 31A-31B illustrate one example of a neuromodulation device 3101 that is similar to that shown in FIGS. 3B-3F. FIG. 31A shows the front of the device, while FIG. 31B shows the back. The neuromodulation device may be used with a gel pad, as shown in FIG. 31C that forms the skin-contacting portion of the electrode, to be worn on the back of the subject's neck.

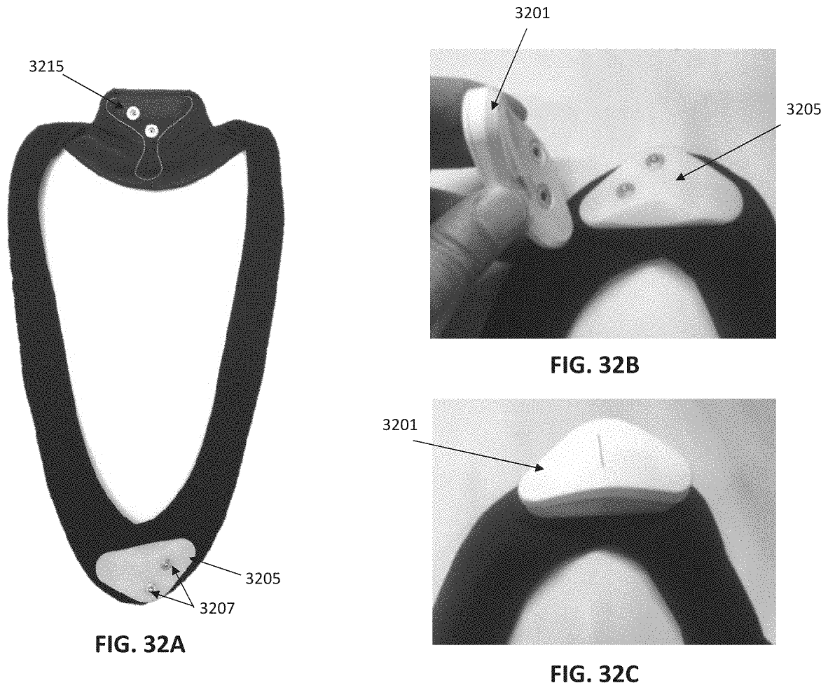

The neuromodulation device is configured to generate a small, pulsed electrical current transmitted through insulated electrical wiring in a neckband (as shown in FIG. 32A-32C) to the electrodes, shown as gel pads such as shown in FIG. 31C. When the gel pads are placed on the back of the subject's neck (i.e., the target anatomy), the hydrogel conducts electrical currents through the skin to modulate nerve bundles in the proximity of the electrode surface, thus providing non-invasive neuromodulation electrical stimulation that may allow autonomic nervous system neuromodulation. The disposable electrode assembly (gel pad) in this example includes a circular area at the apex of the triangular region. It is 3″ wide at the base of the triangle, and 3″ tall, with thickness of approximately 0.1″.

The neuromodulation device in this example contains a power button, an LED indicator, a micro-USB charging port, and a pair of snap connectors to the gel pads. It is recharged using a standard micro-USB connector cable. The device may communicate with a remote (e.g., handheld) device such as a smart phone that has some control and display capabilities.

In the exemplary neuromodulation device shown, a small, rechargeable, embedded 3.7-Volt lithium polymer battery functions as a power source. A battery charging circuit allows the battery to be charged from a power source through a USB Cable. While charging, the neuromodulation device cannot be activated or used.

The neuromodulation device in this example is roughly triangular-shaped, 1.5″ tall and 3″ at the base. The thickness is approximately 0.25″. The maximum current may be limited to 20 mA, and the average current delivery surface area may be about 7.5 cm². The maximum current density may be limited to about 2.6 mA/cm². Any of these neuromodulation devices may include a battery, such as a 3.7V Lithium ion polymer (e.g., 200 mAh).

The neuromodulation device may contain various electrical circuits which function to provide electrical stimulation. An up-conversion circuit raises the 3.7 Volts from the battery to a requisite level for the electrical pulses, i.e., in the range of 30 volts to 65 volts measured at the peak of the pulses. An output voltage and current monitoring circuit monitors the output to assure that the value is within safe bounds. If either the voltage or the current exceeds the set boundary, or if the pulses are longer than a set boundary, this dedicated circuit may shut down the power supply and will not recover unless there is a hardware reset executed by the subject.

In some variations a skin discharge circuit periodically discharges the cumulated charges on the skin through a resistor. This discharge mechanism reduces acute skin sensations coming from the excitation of peripheral nerves when electrical charge accumulates on the skin. A half-bridge circuit may be used to reverse the polarity of the output electrical current on command and provide both positive and negative currents for neuromodulation. An electrical impedance measurement circuit may determine the skin's impedance to allow fine-tuning the amplitude of the electrical current for neuromodulation, to detect if the gel pads have detached from the skin, and/or to verify that the subject has a skin impedance of, e.g., 20 kOhms or less; the device may be configured to require an impedance of below some threshold (e.g., 20 kOhms or less, 15 kOhms or less, 25 kOhms or less, etc.) before allowing delivery of neuromodulation energy.

In the exemplary device shown in FIG. 31B, the neuromodulation device uses two snaps 3107 (electrical connectors) to establish a reliable electrical connection to the electrodes (e.g., the gel pad). The device also includes a micro USB port 3105 and a power button 3103. The output electrical current from the device may go through the snaps to the gel pad(s), which may be on the neckband. In some variations, the neckband includes insulated electrical wires inside the neckband's fabric to conduct the output electrical current to the gel pads.

In FIG. 31C, the disposable gel electrode assembly (gel pad) 3120 may include a hydrogel of high electrical impedance to assure uniform distribution of the electrical current along the gel pad surfaces and to minimize the edge effect at the outer boundary of the electrodes. A silver/silver chloride film underneath the hydrogel may replenish the ions in the gel pad when it is depleted so as to maintain neutral pH at the gel-skin interface to avoid discomforts coming from pH changes during the passage of electrical current through the gel pads. The gel pad assembly may be a disposable gel electrode assembly. The gel pad may also include one or more connectors (e.g., male and/or female snaps, etc.) for electrically the electrodes of the gel pad to the neuromodulation device either directly or through a neckband. In FIG. 31C, the gel pad may include a mechanically flexible base substrate that may be made, e.g., of a PVC film that has silver traces leading from two snaps 3221 to a pair of silver contact surfaces for the gel pad. The snaps may make contact with the neckband to obtain the neuromodulation electrical current.

In FIG. 31C, the electrode pad (e.g., gel pad) has a generally triangular shape which may help define the polarity of the two snaps when connecting to the neuromodulator and/or neckband.

FIG. 32A illustrates one example of a neckband that may be used with an electrode pad (gel pad) and neuromodulator. In FIG. 32A, the neck band is an approximately 10-inch long soft lanyard which may provide a comfortable way to apply and wear the neuromodulation apparatus. In this example, the neckband includes a device platform 3205 that is configured to connect to a neuromodulation device 3201 via two snaps 3207. The snaps may be electrically and mechanically connecting snaps. The snaps may provide mechanical retention of the neuromodulation device to the device platform on the neckband as well as electrical connection. In FIG. 32A, the neckband is shown including a “device platform” 3205 (also referred to herein as a neuromodulation dock) located at the bottom and the electrode attachment “neck” portion on top. FIGS. 32B and 32C illustrate attachment of the neuromodulation device onto the “device platform” region of the neckband.

In the neckband shown in FIGS. 32A-32C, two insulated wires connect the snaps at the device platform 3205 to two snaps on the neck portion 3215 of the neckband. The neuromodulation device fits against the device platform (neuromodulation dock), so that the polarity of the two snaps (e.g., anode, cathode) is assured. This is illustrated in FIGS. 32B-32C, showing the neuromodulation device 3201 connecting to the neuromodulation dock 3205 on the neckband. The neck portion 3215 also includes a roughly triangular alignment guide printed on the neck portion of the neckband 3215 that may match the shape of the disposable gel electrode assembly (gel pad). The electrode assembly (gel pad) may snap onto the two snaps to make electrical contact to the output of the neuromodulation device through the insulated wires inside the neckband. The neck band may also include one or more alignment guides on the neckband to provide directions to the subject (e.g. patient) on the proper attachment of the gel pads and coupling of the gel pads (electrode assembly) to the neck band.

Any of these systems may also optionally include a micro USB to USB charging cable, which may be used to charge the neuromodulation device. In some variations, the neuromodulation device can be charged with any off-the-shelf cable of the same port configuration. For example, the charging source can be any USB port on a computer, or a USB power supply that has 5 Volts+/−10% with electrical current output capability larger than 0.2 Amps.

In some variations these apparatuses may be prescribed (e.g., by physician) to treat a patient suffering from an immune (e.g., autoimmune) disorder, such as psoriasis. The device may be operated with a handheld device (e.g., smartphone, table, computer, etc.). The handheld device may include a processor and memory and may be preloaded with an application software (“app”) for controlling and/or monitoring the delivery of a non-invasive neuromodulation treatment or treatment regimen. For example, an app (e.g., software, firmware, etc. and specifically a set of instructions stored in a memory) may track and/or control the treatment across multiple doses, such as a treatment regimen for treating psoriasis.

In some variations, operation of the neuromodulation system may include the subject first turning on the neuromodulation device by pressing its power button. Activating the device may illuminate the LED indicator (e.g., to display a pulsing white light). The subject may then pair the Neuromodulator to the handheld device (e.g., via Bluetooth) by opening the App and following on-screen instructions of the user interface. The LED indicator may indicate when pairing is successful; for example, the LED may pulse for the duration of pairing and turn to a solid white state when successfully paired. The App may visually notify the subject when pairing is complete.

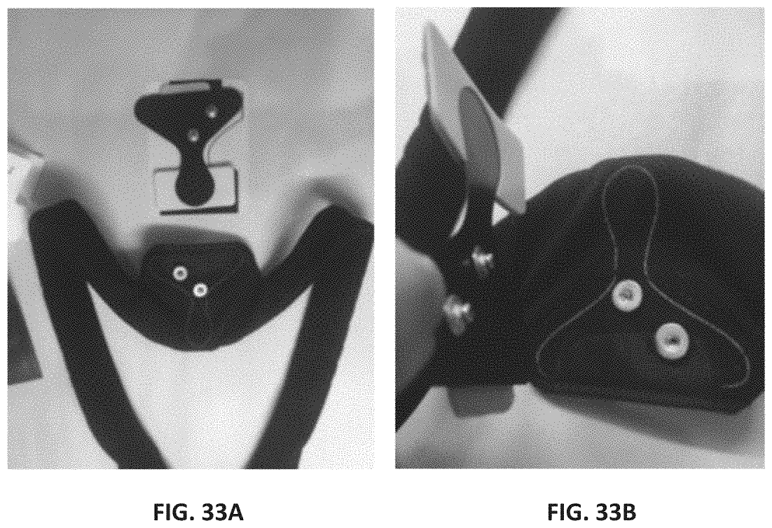

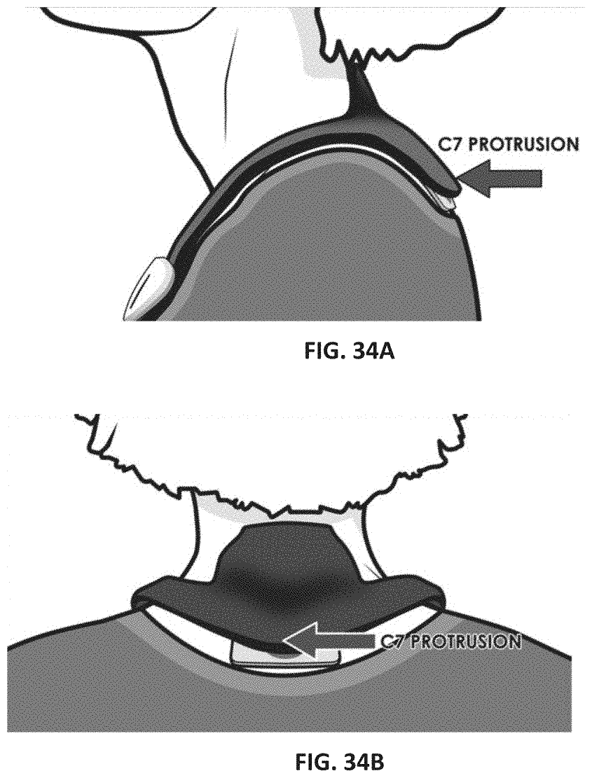

Upon power-on and pairing to the App, the subject (e.g., patient) may then prepare the device for placement by attaching the neuromodulation device to the neckband, as illustrated in FIGS. 32B-32C and described above. This may include aligning the snap-connectors on the back of the neuromodulator to the corresponding platform of the neckband and pressing until a “snap” is heard. The subject may then attach a gel pad (e.g., electrode assembly) to the neckband by pressing together the snap connectors between a non-adhesive side of the gel pad and the corresponding neckband segment, as shown in FIGS. 33A-33B. The subject may then remove the adhesive backing on the gel pad and ready the device for placement on the neck. To place the device, the subject may place the neckband over his/her head, with the adhesive side of the gel pad placed downwards and may affix the bottom of the gel pad at around the C7 Protrusion (e.g., near or on the midline of the neck/back), as shown in FIGS. 34A-34B. The neuromodulator may then rest at the patient's chest, thus the device may be comfortably worn during treatment.

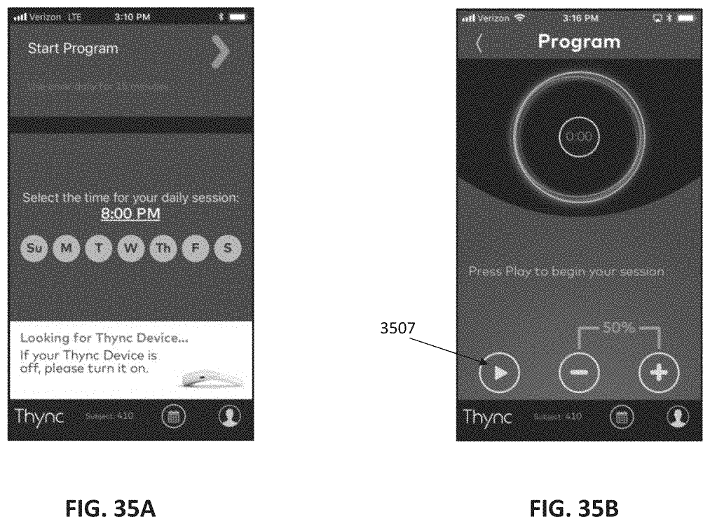

In some variations, the subject can initiate a treatment session by tapping a “start program” control on the app (and/or by pushing the button on the neuromodulator). See, e.g., FIG. 35A. The user interface may allow the user to set the scheduling of the dosing (e.g., when once or twice daily dosing, e.g., 15 minute doses, are used). Alternatively or additionally, the subject may tap a “play button” 3507 on a user interface of the app, as illustrated in FIG. 35B. After waiting (e.g., some duration such as 10 seconds) for session to load and/or start, the subject can adjust the program intensity through the app and/or directly by a control on the neuromodulator and/or neckband. For example, in the user interface shown in FIG. 35B, on-screen “+” and “−” buttons may be used to increase or decrease the intensity. The subject may adjust the intensity of the program until a barely-noticeable “tingling” sensation is felt at the placement site. Each treatment session may last a predetermined time period (e.g., 5 minutes, 10 minutes, 12.5 minutes, 15 minutes, 16 minutes, 17 minutes, 18 minutes, 19 minutes, 20 minutes, etc.). In some variations a minimum treatment time may be used as part of the dosing regimen, such as a minimum treatment time of 15 minutes/day. The subject may end the treatment at any time by tapping the “Stop” control (e.g., on the device and/or the app).

In some variations the subject may not control the intensity and/or the intensity may be set automatically by the apparatus. In some variations the user may only adjust the intensity; the other dosing parameters, including the maximum allowed dose, the waveform parameters (frequency, pulse width, carrier frequency, duty cycle, etc.) is automatically controlled and not subject to user modification. Adjusting the intensity may adjust the peak amplitude of the waveform.

For example, in FIGS. 34A and 34B the user interfaces that may control the neuromodulation device and may initiate and/or terminate treatment. The subject can use the “+” and/or “−” buttons on the app to control the intensity of the neuromodulation within a permitted region of adjustment. The app may provide visual notifications to the subject at the initiation and completion of a treatment cycle, as well as if unexpected device operations occur (e.g., removal of the gel pads, wireless disconnection, low battery status, etc.). The app may also provide optional notifications to the subject to improve compliance by reminding the subject of his/her daily treatment session. In some variations, the subjects may select a daily recurring time for the reminder. To monitor subject compliance, the app may additionally capture device use data. In some variations, the app may verify patients' prescription status. Secure protocols (i.e., AES-256 Encryption) may be used to communicate with the app and the neuromodulation device.

In FIG. 35A, the screen displays an initial user interface screen, displaying push notification and Bluetooth pairing settings. FIG. 35B shows one example of a user interface (e.g., on-screen display) during a treatment session, with the “play” and intensity control buttons displayed on the bottom.

In some variations, a secure web portal may be used to allow a physician to review a subject's device usage and compliance. After the physician signs in and enters a specific subject number, the web portal may display a monthly calendar interface to display usage for the subject. Complete and incomplete sessions may be listed for each day in the calendar and gives a physician a visual snapshot of subject compliance. A similar web portal may similarly allow physicians to track the compliance of each patient under their prescription.

Thus, in any of these variations, the apparatus (e.g., including a set of computer-readable instructions that, when executed by a processor in the apparatus, cause the processor to: set the dosing regimen for treating psoriasis and cause the controller to apply electrical energy for the user-specified dosing regimen) may also report patient compliance and the user may modify the appliance, including in particular the set of instructions, to adjust the dose based on the patient compliance. For example, the set of instructions may include instructions that, when executed on the processor, cause patient compliance data (e.g., actual dose delivery, duration of applied dose, intensity of applied dose, time/date of applied dose, etc.) to be stored and/or transmitted (as acquired, when polled by the user and/or automatically after accumulating a predetermined amount). Similarly, treatment efficacy data may be stored and/or transmitted. For example, treatment efficacy may be determined by the controller (executing the set of instructions) by receiving patient-reported outcome (e.g., patient indications of psoriasis treatment outcome/size and/or number of plaques, etc.), storing and/or transmitting this information.

In any of these variations, the apparatus may be further configured to allow the user to modify the dosing regimen. As mentioned, the user may modify the dosing regimen based on the compliance data. For example, the user (physician, nurse, and/or other healthcare provider) may modify the dose after review of the compliance and/or efficacy data.

In general, the apparatuses described herein may provide low-level electrical stimulation of the cervical and thoracic spinal nerves to systemically modulate autonomic nervous system activity, which may in turn reduce the effects of psoriasis and/or other immune disorders, as illustrated above. Without being bound by any particular theory of operation, the Applicants have proposed that the neural pathways modulated by the application of appropriate and specific non-invasive neuromodulation at the neck are involved in a number of important physiological processes including the stress response, which may affect patients suffering from moderate to severe psoriasis.

Although the exact underlying pathophysiologic mechanism for psoriasis is unclear, stress and its underlying neurologic response have been shown as influencing factors within psoriasis. Specifically, stress, which is characterized by increased autonomic response, is shown to increase levels of substance P, vasoactive intestinal peptide (VIP), and nerve growth factor (NGF) in the skin. Substance P and VIP have stimulatory effects on keratinocytes and upregulate proteins such as tumor necrosis factor-alpha (TNFa), interleukin (IL)-1 and IL-8, which are implicated in chronic inflammation. Levels of substance P and VIP are significantly upregulated in psoriasis and have been shown to be critical in the initiation, as well as maintenance, of the disease process. There is also substantial evidence to support NGF, a neuropeptide involved in maintenance, proliferation, and survival of neurons, as an important contributor to the pathophysiology of psoriasis. Studies have shown that keratinocytes in patients with psoriasis are programmed to produce increased levels of NGF, which cause inflammatory changes in the skin favoring de-differentiation and epidermal hyperproliferation. In addition, elevated levels of NGF have been shown to trigger release of histamine by mast cells and proliferation of cutaneous lymphocytes. Interestingly, there have been several case reports in which psoriasis patients with nerve damage have exhibited unilateral local improvement and even complete remission of their psoriasis in the denervated dermatomal region.

While the current FDA-approved or cleared treatments for psoriasis include topical therapies, phototherapy, oral systemic immunosuppressive agents, and biologic injectable agents, limited studies have also shown stress reduction to be an effective adjunct treatment option for patients who are stress-responders. Both psychotherapy and pharmacotherapy appear to be effective at reducing stress and improving psoriasis severity. It is possible that reducing stress may help all patients with psoriasis.

Another method of stress reduction is through neuromodulation of noradrenergic activity. The neuromodulation mechanism described herein have been shown to suppress psychophysiological and biochemical stress responses in humans under various experimental conditions. Subjects treated with non-invasive neuromodulation reported significantly lower levels of tension and anxiety on the “Profile of Mood States” scale compared to placebo. Furthermore, when subjects were experimentally stressed, non-invasive neuromodulation produced a significant suppression of heart rate variability, galvanic skin conductance, and salivary α-amylase levels compared to placebo. Collectively, these observations demonstrated that non-invasive neuromodulation can dampen basal sympathetic tone, as well as attenuate sympathetic activity in response to acute stress induction.

The use of the non-invasive neuromodulation device has not been associated with serious adverse events and common side effects are primarily limited to local skin reactions including skin tingling, itching, and mild burning sensations. The method described herein typically use KHz carrier waveforms that are burst at lower frequencies (e.g., <200 Hz). These frequency regimes may depolarize nerves below the skin surface and reliably activate nerve fibers. Pain from non-invasive neuromodulation electrical stimulation may be caused by charge buildup in the skin (i.e., the skin acting as a capacitor) and pH changes at the surface. The apparatus and systems described herein may buffer pH changes at the skin using medical grade hydrogel electrodes that contain Ag/AgCl sacrificial anode/cathode layers.

In use a treatment may include the application of a non-invasive neuromodulation waveform (or combination of waveforms) including a pulsed biphasic current (e.g., 1-11 kHz; 20-50% duty cycle), having an average amplitude of between 1-7 mA. This treatment may be, e.g., 15 minutes or more a day. The Applicants have found that the frequency range and duty cycle, as well as the dosing regimen (as discussed above) may be important in achieving robust treatment of psoriasis. For example, the near-identical application of non-invasive neuromodulation waveforms having a frequency range of, for example, 1-3 kHz (e.g., at 15% duty cycle) also having an average amplitude of between 1-7 mA for 15 minutes may not result in as robust (if any) treatment of psoriasis, although the skin sensation during the delivery of the waveform may be identical to the high duty-cycle and frequency treatments. Thus, in any of the methods and apparatuses described herein, duty cycle of stimulation may be greater than 20% (e.g., between 20-99%, between 20-90%, between 20-80%, between 20-70%, between 20-60% between 20-50%, etc.) when the frequency is greater than 1 kHz (e.g., between 1-60 kHz, between 1-50 kHz, between 1-40 kHz, between 1-30 kHz, between 1-20 kHz, between 1-15 kHz, between 1-14 kHz, between 1-13 kHz, between 1-12 kHz, between 1-11 kHz, etc.). The apparatuses described herein may be specifically configured to provide non-invasive neuromodulation output within these ranges. The dosing regimen may include 5× weekly or more (e.g., 6× weekly, 7× weekly, etc.) including daily dosing; the dosing may be consecutive (e.g., every day for x or more days, where x is, for example 63 days, 70 days, 77 days, 84 days, 91 days, 100 days, etc.)

### Waveforms

As discussed above, effective psoriasis treatment may depend on the treatment protocol, including the location (e.g., behind the neck placement, particularly between the proper regions), the repeated (e.g., >3× weekly, >4× weekly, >5× weekly, etc.) and consistent use of the stimulation and the stimulation waveforms used.

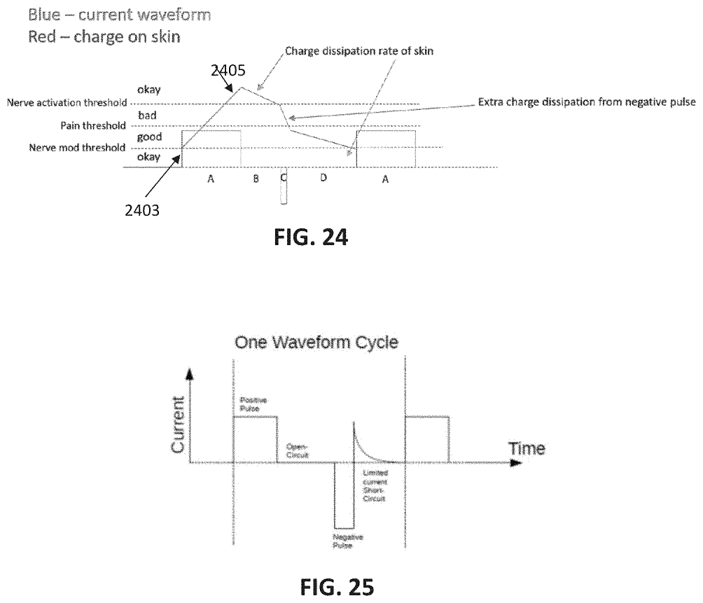

For example, the methods and apparatuses described herein may generally use high frequency (e.g., KHz) carrier waveforms that are burst at lower frequencies (<200 Hz). High frequencies may be used because they allow for the delivery of high peak amplitude currents (7-20 mA) without substantial induced pain. High amplitude currents are helpful for penetration across tissue and consistent activation of nerve fibers. Pain from non-invasive neuromodulation electrical stimulation may be caused by charge buildup in the skin (e.g., the skin acts as a capacitor) and pH changes at the surface. A simplified diagram of this concept is shown in FIG. 24. In this example, a single bipolar “pulse” 2403 that forms the basic unit of the pulsing regime is shown, which may then repeat; it includes regions A, B, C, and D. The positive pulse is in region A, the negative pulse in region C. Regions B and D are quiescent, but the charge may discharge in region D. Region A corresponds to a positive pulse that raises charge above the nerve activation threshold (which may restore nerve activation). The line 2405 in FIG. 24 shows the charge accumulated during the pulse. The charge stays above the nerve activation level for some time (slightly less than region B) to activate the nerve. Once the nerve is activated, a negative-going pulse (region C) is use to reduce charge below the pain threshold (shown on left side of figure). This pulse may be short enough that the nerve continues to be modulated (e.g., the charge dissipated is not above the nerve modulation threshold). In region D, the nerve continues to be effectively modulated until the end of the single pulse cycle, which is then repeated.

Applying charge to the skin may change the skin pH, which may also lead to pain. The methods described herein may buffer pH changes at the skin using custom electrodes. The charge buildup at the skin may be mitigated through the use of negative pulses including a “short-circuiting pulse”. This is shown in FIG. 25.

The DC component consists of the positive pulses which depolarize the axon membrane. The high peak currents allow for depolarization of nerves at greater depths. The key is to have an adequate charge per phase which means that enough charge is passing through a given time to adequately depolarize nerves at a certain depth.

This may be described as charge per DC phase (e.g., microcoulombs per phase) and may be equal to:

Charge per DC phase=Current (mA)*Duration of positive current phase (ms) (1)

The duration of positive current phase (see, e.g., FIG. 1, tp) may be equal to:

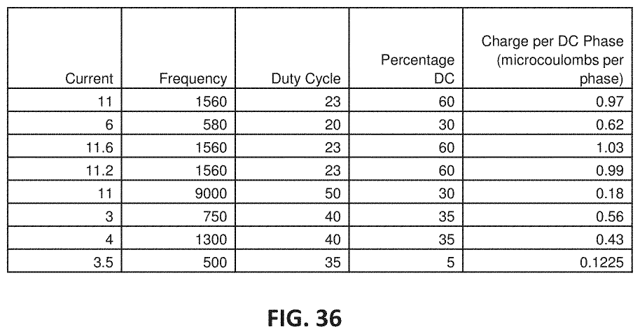

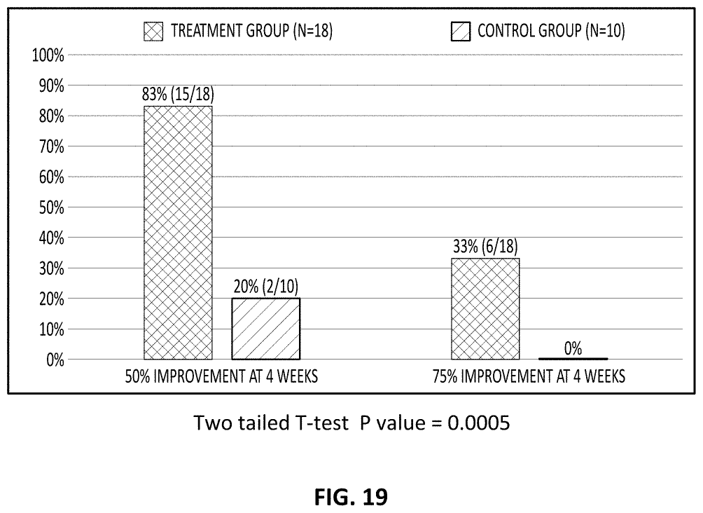

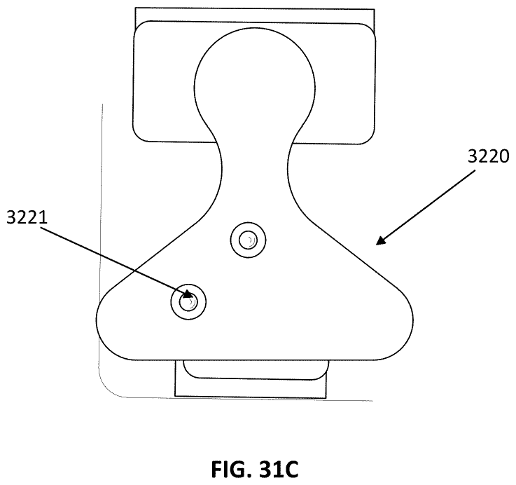

Duration of positive current phase=% DC/100*% Duty/100*1000/Frequency (2)

Where % DC is the DC percentage, and the % Duty is the duty cycle percentage. FIG. 36 is a table illustrating examples of charge per DC phase (e.g., microcoulombs per phase) for a variety of waveform parameters that may be used treat psoriasis, for example, In general these waveforms may have a charge per phase of between 0.1-10 μC/phase (microcoulombs) per phase (e.g., between about 0.1-9 μC/phase, between about 0.1-8 μC/phase, between about 0.1-7 μC/phase, between about 0.1-6 μC/phase, between about 0.1-5 μC/phase, between about 0.1-4 μC/phase, between about 0.1-3 μC/phase, between about 0.1-2 μC/phase, between about 0.2-5 μC/phase, between about 0.2-3 μC/phase, etc.). The charge per DC phase may be determined at the maximum (or full) available intensity that may be applied to the patient. In some variations, the patient may adjust the intensity. The charge per phase may be determined from the waveform provided by the device that may be adjusted by the patient. For example, a patient may adjust the intensity to be an intensity of between about 50-80% of the available waveform intensity. Thus the actual charge per DC phase may be approximately between about 0.5 to about 0.8 μC/phase, within the broader range of about 0.1-10 μC/phase (or any of the sub-ranged listed above). As mentioned, outside of these charge/phase ranges, the waveform may not work to treat the inflammatory disorder, including psoriasis.

Thus, described herein are methods of treating a patient for an inflammatory disorder (e.g., methods of treating psoriasis) by non-invasively applying electrical energy comprising non-invasively applying electrical energy to the subject to reduce one or more of the size and number of psoriasis plaques, wherein the applied electrical energy has charge/phase (in μC/phase) of between about 0.1 and 10 μC/phase (e.g., between 0.1-10 μC/phase (microcoulombs) per phase (e.g., between about 0.1-9 μC/phase, between about 0.1-8 μC/phase, between about 0.1-7 μC/phase, between about 0.1-6 μC/phase, between about 0.1-5 μC/phase, between about 0.1-4 μC/phase, between about 0.1-3 μC/phase, between about 0.1-2 μC/phase, between about 0.2-5 μC/phase, between about 0.2-3 μC/phase, etc.).

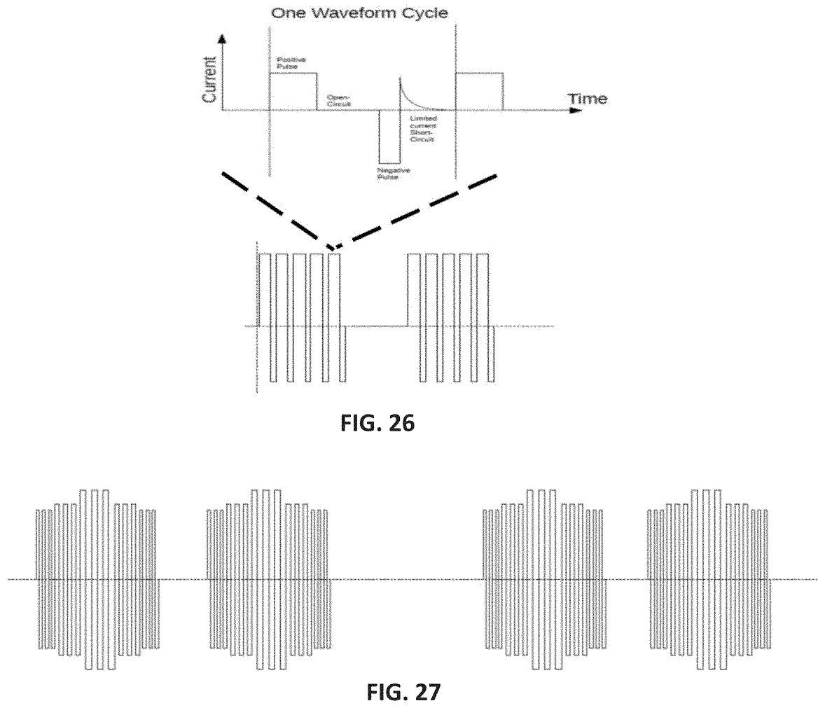

This high frequency waveform may then be burst at lower frequencies which are more relevant to nerve stimulation. Constant current may be a very ineffective method of activating nerves as this leads to inconsistent activation. The resulting waveform is shown in FIG. 26. By shaping the amplitude of the bursts, even higher peak amplitudes may be achieved as shown in FIG. 27.

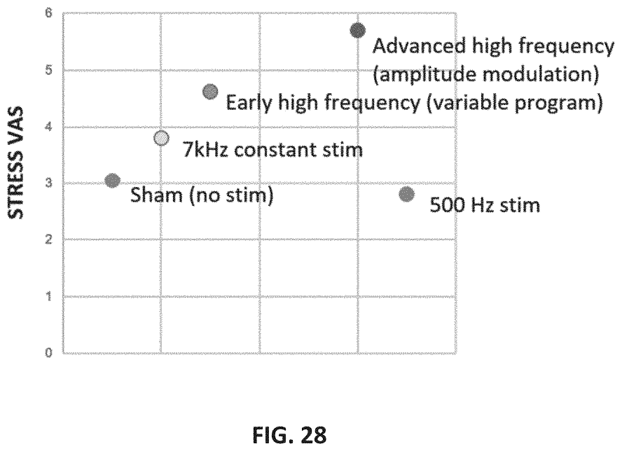

A stress response may be used as a measure of sympathetic activity to develop these algorithms through the testing of thousands of subjects. This data has shown that only a specific subset of stimulation waveforms are effective. Note that a 500 HZ stimulation does show any effect (similar to or worse than a sham, with no stimulation). FIG. 28 illustrates this effect. FIG. 29 describes the stimulation parameters for an effective waveform. In contrast, FIG. 30 illustrates a sham waveform.

Any of the treatment methods and treating regimes described herein may use autonomic measurements for feedback. In some variations, autonomic measurements may be used to predict whether someone will be a responder to our neuromodulation treatment. As an example, a patient's autonomic activity may be measured acutely in the doctor's office in response to our neuromodulation program before starting treatment. If the patient has a certain level of change to autonomic nervous system (ANS) activity as a result of our stimulation, they may be deemed as a good candidate for treatment. If ANS activity does not change acutely, other neuromodulation programs may be tested and if none work the patient may be considered as not a good candidate for treatment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A wearable electrical energy applicator apparatus configured to treat psoriasis by the delivery of electrical energy, the apparatus comprising:
a first electrode;
a second electrode;
a controller configured to apply an electrical waveform between the first and second electrodes, wherein the waveform has a peak amplitude of greater than 3 mA, a frequency of greater than 250 Hz, and a duty cycle of greater than 15%; and
a computer readable medium having a set of computer-readable instructions recorded thereon, the computer-readable instructions, when executed by a processor, cause the processor to: implement a set dosing regimen for treating psoriasis causing the controller to apply the electrical waveform at the dosing regimen spanning a plurality of days.

2. The apparatus of claim 1, wherein the first and second electrode are adapted to be worn along the midline of a back of a neck.

3. The apparatus of claim 1, wherein the apparatus further comprises a neckband configured to be worn around a neck, wherein the neckband comprises an electrode alignment guide configured to couple to the first and second electrode and a dock configured to couple to the controller and an electrical connection between the electrode alignment guide and the dock.

4. The apparatus of claim 1, wherein the dosing regimen is configured to apply electrical energy at least once per day for at least 10 minutes each day, each of 5 or more days a week for at least four weeks.

5. The apparatus of claim 1, wherein the dosing regimen is configured to apply electrical energy at least once per day for at least 15 minutes each day for at least eight weeks.

6. The apparatus of claim 1, wherein the controller is enclosed in a housing having two or more electrical connectors configured to electrically connect to the first and second electrodes.

7. The apparatus of claim 1, wherein the controller is enclosed in a housing weighing less than 50 g.

8. The apparatus of claim 1, wherein the controller is configured to apply electrical energy having a peak amplitude of greater than 3 mA, a frequency of greater than 1 kHz, and a duty cycle of 20% or more.

9. The apparatus of claim 1, wherein the controller is further configured to prevent the device from delivering electrical energy at 15% duty cycle or less.

10. The apparatus of claim 1, wherein the computer readable medium is configured to operate on a smartphone.

11. The apparatus of claim 1, further comprising a wireless communication circuit configured to wirelessly communicate between the controller and the processor executing the computer-readable instructions.

12. The apparatus of claim 1, further wherein the controller is configured restrict the applied electrical waveform to an electrical waveform having a charge per phase of between about 0.1 and 10 μC/phase.

13. The apparatus of claim 1, wherein the computer-readable instructions are further configured to transmit compliance and/or efficacy data from the apparatus to a remote server for review by a physician.
